(19) United States
(12) Patent Application Publication
(10) Pub. No.: US 2007/0265270 A1
(43) Pub. Date: Nov. 15, 2007

(54) Cinnoline derivatives as phosphodiesterase 10 inhibitors

(76) Inventors: Stephen A. Hitchcock, Westlake Village, CA (US); Ruiping Liu, Huntington, NY (US); Mark P. Arrington, Westwood, NJ (US); Allen T. Hopper, Mahwa, NJ (US); Richard D. Conticello, Cortlandt Manor, NY (US); Truc M. Nguyen, New York, NY (US); M. Diana Danca, Mendham, NJ (US); Carla M. Gauss, White Plains, NY (US)

Correspondence Address: JONES DAY, 222 EAST 41ST ST, NEW YORK, NY, 10017, US

(21) Appl. No.: 11/708,868

(22) Filed: Feb. 20, 2007

Related U.S. Application Data
(60) Provisional application No. 60/774,656, filed on Feb. 21, 2006.
(60) Provisional application No. 60/775,794, filed on Feb. 23, 2006.

(51) Int. Cl. A61K 31/502; A61K 31/5375; A61P 25/00; A61P 25/18; A61P 3/00; C07D 237/28; C07D 295/00

(52) U.S. Cl. 514/239.2; 514/248; 544/174; 544/237

(57) ABSTRACT

The present invention is directed to certain cinnoline compounds that are PDE10 inhibitors, pharmaceutical compositions containing such compounds and process for preparing such compounds. The invention is also directed to methods of treating diseases treatable by modulation of PDE10 enzyme, such as obesity, non-insulin dependent diabetes, schizophrenia, bipolar disorder, obsessive-compulsive disorder, and the like.

### CROSS-REFERENCE

This application claims the benefit of U.S. Patent Applications Nos. 60/774,656, filed on Feb. 21, 2006, and 60/775,794, filed on Feb. 23, 2006, the disclosures of which are incorporated herein by reference in their entirety.

### FIELD OF THE INVENTION

The present invention is directed to certain cinnoline compounds that are PDE10 inhibitors, pharmaceutical compositions containing such compounds and processes for preparing such compounds. This invention is also directed to methods of treating diseases treatable by inhibition of PDE10 enzyme, such as obesity, non-insulin dependent diabetes, schizophrenia, bipolar disorder, obsessive-compulsive disorder, and the like.

### BACKGROUND

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activity.

Cyclic nucleotides are produced from the actions of adenylyl cyclase and guanylyl cyclase which convert ATP to cAMP and GTP to cGMP. Extracellular signals, often through the actions of G protein-coupled receptors, regulate the activity of the cyclases. Alternatively, the amount of cAMP and cGMP may be altered by regulating the activity of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE 1 is stimulated by Ca²⁺/calmodulin. PDE 2 activity is stimulated by cGMP. PDE 3 is inhibited by cGMP. PDE 4 is cAMP specific and is specifically inhibited by rolipram. PDE 5 is cGMP-specific. PDE6 is expressed in retina.

PDE10 sequences were first identified by using bioinformatics and sequence information from other PDE gene families (Fujishige et al., J. Biol. Chem. 274:18438-18445, 1999; Loughney et al., Gene 234:109-117, 1999; Soderling et al., Proc. Natl. Acad. Sci. USA 96:7071-7076, 1999). The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because of at least three alternative exons encode N termini and two exons encode C termini. PDE10A1 is a 779 amino acid protein that hydrolyzes both cAMP and cGMP. The Kₘ values for cAMP and cGMP are 0.05 and 3.0 micromolar, respectively. In addition to human variants, several variants with high homology have been isolated from both rat and mouse tissues and sequence banks.

PDE10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are enriched in PDE10. Western blots did not reveal the expression of PDE10 in other brain tissues, although immunprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE10 enzyme, for example, in neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

### SUMMARY OF THE INVENTION

In one aspect, provided herein is a compound of Formula (I): 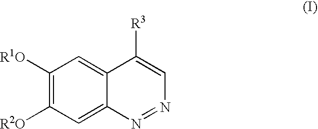
wherein:
R¹ and R² are independently hydrogen, alkyl, or haloalkyl; and
R³ is:
   (i) a ring of formula (a) 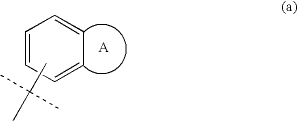
   where A is a monocyclic five-, six-, or seven membered heterocyclyl ring and the ring of formula (a) is substituted with:
   R⁴where R⁴ is hydrogen, cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —NR⁸CO—, —CONR⁹—, —NR¹⁰—, —S—, —SO—, —SO₂—, —NR¹¹SO₂—, or —SO₂NR¹²—, where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
   R⁵ and R⁶ where R⁵ and R⁶ are independently hydrogen, alkyl, cycloalkyl, cycloalkylalkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl;
   and wherein the aromatic or alicyclic ring in R⁴, R⁵, R⁶, and R⁷ is optionally substituted with one to three substitutents independently selected from R^{a}, R^{b}, and R^{c} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are independently hydrogen or fluoro;
   (ii) a ring of formula (b) or (c): 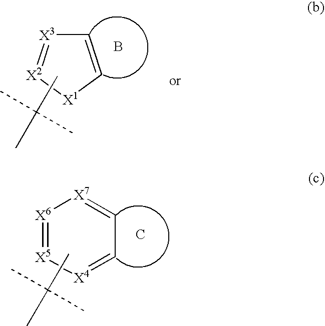
      where:
      X¹, X², and X³ are independently carbon, nitrogen, oxygen or sulfur provided that at least two of X¹, X², and X³ are other than carbon;
      X⁴, X⁵, X⁶ and X⁷ are independently carbon or nitrogen provided that at least two of X⁴, X⁵, X⁶ and X⁷ are other than carbon; and
      B and C are phenyl, a five- or six-membered heteroaryl ring (wherein the five-membered heteroaryl ring contains one or two heteroatoms independently selected from nitrogen, oxygen, and sulfur and the six-membered heteroaryl ring contains one or two nitrogen atoms, the rest of the ring atoms being carbon), or a monocyclic five-, six-, or seven-membered heterocyclyl ring; and
      wherein rings of formulae (b) and (c) are substituted with:
      R¹³ where R¹³ is hydrogen, cycloalkyl, cycloalkylalkyl, aryl, heteroaryl, heterocyclyl, aralkyl, heteroaralkyl, heterocyclylalkyl, or —XR¹⁶ (where X is —O—, —CO—, —C(O)O—, —NR¹⁷CO—, —CONR¹⁸—, —NR¹⁹—, —S—, —SO—, —SO₂—, —NR²⁰SO₂—, or —SO₂NR²¹— where R¹⁷, R¹⁸, R¹⁹, R²⁰ and R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl); and
      R¹⁴ and R¹⁵ where R¹⁴ and R¹⁵ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and
      wherein the aromatic or alicyclic ring in R¹³, R¹⁴, R¹⁵, and R¹⁶ is optionally substituted with one to three substitutents independently selected from R^{f}, R^{g}, and R^{h} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkylcarbonyl, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁱ and R^{j} where Rⁱ and R^{j} are independently hydrogen or fluoro;
   (iii) a monocyclic six- or seven-membered heterocyclyl ring substituted with:
      R²² where R²² is cycloalkyl, aryl, heteroaryl, heterocyclyl, aralkyl, heteroaralkyl, heterocyclylalkyl, or —XR²⁵ (where X is —O—, —CO—, —C(O)O—, —NR²⁶CO—, —CONR²⁷—, —NR²⁸—, —S—, —SO—, —SO₂—, —NR²⁹SO₂—, or —SO₂NR³⁰— where R²⁶, R²⁷, R²⁸, R²⁹ and R³⁰ are independently hydrogen, alkyl, hydroxyalkyl, alkoxyalkyl, aryl, aralkyl, heteroaryl, heteroaralkyl, acyl, or heterocyclylalkyl and R²⁵ is cycloalkyl, cycloalkylalkyl, aryl, heteroaryl, heterocyclyl, aralkyl, heteroaralkyl, or heterocyclylalkyl); and
      R²³ and R²⁴ where R²³ and R²⁴ are independently hydrogen, alkyl, cycloalkyl, cycloalkylalkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkoxycarbonylalkyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and
      wherein the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, amino, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are independently hydrogen or fluoro; or
   (iv) pyrrolidinyl, 2-oxopyrrolidinyl, or 2,4-dioxoimidazolidinyl substituted with:
      R³¹ where R³¹ is aryl, heteroaryl, heterocyclyl, aralkyl, heteroaralkyl, heterocyclylalkyl, or —XR³⁴ (where X is —O—, —CO—, —C(O)O—, —NR³⁵CO—, —CONR³⁶—, —NR³⁷—, —S—, —SO—, —SO₂—, —NR³⁸SO₂—, or —SO₂NR³⁹— where R³⁵, R³⁶, R³⁷, R³⁸ and R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl); and
      R³² and R³³ where R³² and R³³ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, aryl, heteroaryl or heterocyclyl; and
      wherein the aromatic or alicyclic ring in R³¹, R³², R³³, and R³⁴ is optionally substituted with one to three substitutents independently selected from R^{p}, R^{q}, and R^{r} which are independently alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, sulfonyl, aminocarbonyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{s} and R^{t} where R^{s} and R^{t} are independently hydrogen or fluoro.

In some embodiments, provided herein is a compound of Formula (I) as described above, or an individual stereoisomer, mixtures of stereoisomers, or a pharmaceutically acceptable salt of the compound of Formula (I), provided that:
(i) the compound of Formula (I) is not 4-(4-(3-chlorophenyl)piperazin-1-yl)-6,7-dimethoxycinnoline; 4-(4-(benzo[d]isothiazol-3-yl)piperazin-1-yl)-6,7-dimethoxycinnoline;
(ii) when R³ is pyrrolidin-1-yl, R³¹ is not —XR³⁴ where X is —O— and R³⁴ is substituted or unsubstituted aryl or heteroaryl;
(iii) when R³ is piperidin-1-yl, one of R²³ and R²⁴ is hydrogen, and R²² is substituted or unsubstituted aryl or heteroaryl, then the other of R²³ and R²⁴ is not hydrogen, alkyl, carboxy, alkoxycarbonyl, cyano, hydroxyl, alkoxy, —COR, —CONRR′ or —NRR′ (where R and R′ are independently hydrogen, alkyl, or unsubstituted aryl), or —NHCOR (where R is alkyl or unsubstituted aryl); and
(iv) when R³ is piperidin-1-yl, R²³ and R²⁴ are both hydrogen or one of R²³ and R²⁴ is hydrogen and the other of R²³ and R²⁴ is substituted or unsubstituted aryl or heteroaryl, then R²² is not —COR²⁵ (where R²⁵ is unsubstituted aryl), —COOR²⁵ (where R²⁵ is unsubstituted aryl), —CONR²⁵R²⁷, —NR²⁵R²⁸ or —NHCOR²⁵ (where R²⁷ and R²⁸ are hydrogen, alkyl, or unsubstituted aryl, and each R²⁵ is unsubstituted aryl).

In some embodiments, provided herein is a compound of Formula (I) as described above, or an individual stereoisomer, mixtures of stereoisomers, or a pharmaceutically acceptable salt thereof, provided that:
(i) the compound of Formula (I) is not 4-(4-(3-chlorophenyl)piperazin-1-yl)-6,7-dimethoxycinnoline; 4-(4-(benzo[d]isothiazol-3-yl)piperazin-1-yl)-6,7-dimethoxycinnoline;
(ii) when R³ is pyrrolidin-1-yl, R³¹ is not —XR³⁴ where X is —O— and R³⁴ is substituted or unsubstituted aryl or heteroaryl;
(iii) when R³ is piperidin-1-yl, one of R²³ and R²⁴ is hydrogen, and R²² is substituted or unsubstituted aryl or heteroaryl, then the other of R²³ and R²⁴ is not hydrogen, alkyl, carboxy, alkoxycarbonyl, cyano, hydroxyl, alkoxy, —COR, —CONRR′ or —NRR′ (where R and R′ are independently hydrogen, alkyl, or substituted or unsubstituted aryl), or —NHCOR (where R is alkyl or substituted or unsubstituted aryl); and
(iv) when R³ is piperidin-1-yl, R²³ and R²⁴ are both hydrogen or one of R²³ and R²⁴ is hydrogen and the other of R²³ and R²⁴ is substituted or unsubstituted aryl or heteroaryl, then R²² is not —COR²⁵ (where R²⁵ is substituted or unsubstituted aryl), —COOR²⁵ (where R²⁵ is substituted or unsubstituted aryl), —CONR²⁵R²⁷, —NR²⁵R²⁸ or —NHCOR²⁵ (where R²⁷ and R²⁸ are hydrogen, alkyl, or substituted or unsubstituted aryl, and each R²⁵ is substituted or unsubstituted aryl).

In one aspect, this invention is directed to a pharmaceutical composition comprising a compound of Formula (I) or a pharmaceutically acceptable salt thereof and a pharmaceutically acceptable excipient.

In another aspect, this invention is directed to a method of treating a disorder treatable by inhibition of PDE10 in a patient which method comprises administering to the patient a pharmaceutical composition comprising a compound of Formula (I) or a pharmaceutically acceptable salt thereof and a pharmaceutically acceptable excipient. Within this aspect, the disease is obesity, non-insulin dependent diabetes, Huntington's disease, schizophrenia, bipolar disorder, or obsessive-compulsive disorder.

It will be readily apparent to a person skilled in the art that the pharmaceutical composition could contain one or more compounds of Formula (I) (including individual stereoisomer, mixtures of stereoisomers where the compound of Formula (I) has at least one stereochemical center), a pharmaceutically acceptable salt thereof, or mixtures thereof.

### DETAILED DESCRIPTION

Definitions

Unless otherwise stated, the following terms used in the specification and claims are defined for the purposes of this Application and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring, e.g, cycloalkyl or heterocyclyl ring.

“Alkylene” means a linear or branched saturated divalent hydrocarbon radical of one to six carbon atoms unless otherwise stated, e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means an —OR radical where R is alkyl as defined above, e;g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Alkoxycarbonylalkyl” means an -(alkylene)-C(O)OR radical where R is alkyl as defined above, e.g., methoxycarbonylmethyl, ethoxycarbonylethyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means a —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, and R′ is hydrogen, alkyl, hydroxyalkyl, alkoxyalkyl, aryl, aralkyl, heteroaryl, heteroaralkyl, or haloalkyl, each as defined herein, e.g, aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means an —OR radical where R is aminoalkyl as defined above, e.g., 2-aminoethoxy, 2-dimethylaminopropoxy, and the like.

“Aminocarbonyl” means a —CONRR′ radical where R is hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —CONH₂, methylaminocarbonyl, 2-dimethylaminocarbonyl, and the like.

“Aminosulfonyl” means a —SO₂NRR′ radical where R is hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —SO₂NH₂, methylaminosulfonyl, 2-dimethylaminosulfonyl, and the like.

“Aminosulfinyl” means a —SONRR′ radical where R is hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —CONH₂, methylaminosulfinyl, 2-dimethylaminosulfinyl, and the like.

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetyl, propionyl, benzoyl, pyridinylcarbonyl, and the like. When R is alkyl, the radical is also referred to herein as alkylcarbonyl.

“Acylamino” means a —NHCOR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetylamino, propionylamino, and the like.

“Aryl” means a monovalent monocyclic or bicyclic aromatic hydrocarbon radical of 6 to 12 ring atoms e.g., phenyl, naphthyl or anthracenyl.

“Aralkyl” means an -(alkylene)-R radical where R is aryl as defined above.

“Cycloalkyl” means a cyclic saturated monovalent bridged or non-bridged hydrocarbon radical of three to ten carbon atoms, e.g., cyclopropyl, cyclobutyl, cyclopentyl, cyclohexyl, or adamantyl, and the like.

“Cycloalkylalkyl” means an -(alkylene)-R radical where R is cycloalkyl as defined above; e.g., cyclopropylmethyl, cyclobutylmethyl, cyclopentylethyl, or cyclohexylmethyl, and the like.

“Cycloalkyloxy” means an —OR radical where R is cycloalkyl as defined, e.g., cyclopropyloxy, cyclobutyloxy, cyclopentyloxy, cyclohexyloxy, and the like.

“Cycloalkylalkyloxy” means an —OR radical where R is cycloalkylalkyl as defined above, e.g., cyclopropylmethyloxy, cyclobutylmethyloxy, cyclopentylethyloxy, cyclohexylmethyloxy, and the like.

“Carboxy” means —COOH.

“Disubstituted amino” means a —NRR′ radical where R and R′ are independently alkyl, acyl, sulfonyl, aryl, aralkyl, heteroaryl, or heteroaralkyl, each as defined herein defined above, e.g., dimethylamino, phenylmethylamino, and the like. When R and R′ are alkyl, the —NRR′ radical may are also be referred to herein as dialkylamino.

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₂, and the like.

“Haloalkoxy” means an —OR radical where R is haloalkyl as defined above, e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means an —OR radical where R is hydroxyalkyl as defined above.

“Heterocyclyl” means a saturated or unsaturated monovalent monocyclic group of 3 to 8 ring atoms in which one or two ring atoms are heteroatom independently selected from N, O, and S(O)ₙ, where n is an integer from 0 to 2, the remaining ring atoms being C. Additionally, one or two ring carbon atoms can optionally be replaced by a —CO— group and the heterocyclic ring may be fused to phenyl or heteroaryl ring, provided that the heterocyclic ring is not aromatic. Unless stated otherwise, the fused heterocyclyl ring can be attached at any ring atom. More specifically the term heterocyclyl includes, but is not limited to, pyrrolidino, piperidino, 2-oxopyrrolidinyl, 2-oxopiperidinyl, morpholino, piperazino, tetrahydropyranyl, thiomorpholino, and the like. When the heterocyclyl ring has five, six or seven ring atoms and is not fused to phenyl or heteroaryl ring, it may be referred to herein as “monocyclic five- six-, or seven membered heterocyclyl ring or five- six-, or seven membered heterocyclyl ring”. When the heterocyclyl ring is unsaturated it can contain one or two double bonds provided that the ring is not aromatic.

“Heterocyclylalkyl” means an -(alkylene)-R radical where R is heterocyclyl ring as defined above, e.g., tetraydrofuranylmethyl, piperazinylmethyl, morpholinylethyl, and the like.

“Heteroaryl” means a monovalent monocyclic or bicyclic aromatic radical of 5 to 10 ring atoms where one or more, preferably one, two, or three, ring atoms are heteroatom independently selected from N, O, or S, the remaining ring atoms being carbon.

“Heteroaralkyl” means an -(alkylene)-R radical where R is heteroaryl as defined above.

“Methylenedioxy” means —O—CH₂—O—.

“Monosubstituted amino” means an —NHR radical where R is alkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., methylamino, 2-phenylamino, hydroxyethylamino, and the like.

“Oxo” means ═(O) group.

The present invention also includes prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1999, the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include, for instance, acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In certain embodiments, a “pharmaceutically acceptable salt” can include, for instance, salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that the pharmaceutically acceptable salts are, in general, non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof are within the scope of this invention. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) are within the scope of this invention.

“Optional” or “optionally” means that the subsequently described event or circumstance may but need not occur, and that the description includes instances where the event or circumstance occurs and instances in which it does not. For example, “heterocyclyl group optionally mono- or di-substituted with an alkyl group” means that the alkyl may but need not be present, and the description includes situations where the heterocyclyl group is mono- or disubstituted with an alkyl group and situations where the heterocyclyl group is not substituted with the alkyl group.

“Optionally substituted phenyl” means a phenyl ring optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, sulfinyl, and sulfonyl, each as defined herein.

“Optionally substituted heteroaryl” means a monovalent monocyclic or bicyclic aromatic radical of 5 to 10 ring atoms where one or more, preferably one, two, or three, ring atoms are heteroatoms independently selected from N, O, and S, the remaining ring atoms being carbon that is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, or carboxy, cycloalkyl, cycloalkylalkyl, cycloalkoxy, cycloalkylalkyloxy, sulfinyl, and sulfonyl, each as defined herein. More specifically the term optionally substituted heteroaryl includes, but is not limited to, pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl, substituted or unsubstituted as indicated above.

“Optionally substituted heterocyclyl” means a saturated or unsaturated monovalent cyclic group of 3 to 8 ring atoms in which one or two ring atoms are heteroatoms independently selected from N, O, and S(O)ₙ, where n is an integer from 0 to 2, the remaining ring atoms being C. One or two ring carbon atoms can optionally be replaced by a —CO— group and is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, or carboxy, cycloalkyl, cycloalkylalkyl, cycloalkoxy, cycloalkylalkyloxy, sulfinyl, and sulfonyl, each as defined herein. More specifically the term optionally substituted heterocyclyl includes, but is not limited to, optionally substituted pyrrolidino, piperidino, morpholino, piperazino, tetrahydropyranyl, and thiomorpholino, substituted or unsubstituted as indicated above.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfinyl” means a —SOR radical where R is alkyl, haloalkyl, aryl, aralkyl, heteroaryl, heteroaralkyl, heterocyclyl, or heterocyclylalkyl, each as defined above, e.g., methylsulfinyl, phenylsulfinyl, benzylsulfinyl, pyridinylsulfinyl, and the like.

“Sulfonyl” means a —SO₂R radical where R is alkyl, haloalkyl, aryl, aralkyl, heteroaryl, heteroaralkyl, heterocyclyl, or heterocyclylalkyl, each as defined above, e.g., methylsulfonyl, phenylsulfonyl, benzylsulfonyl, pyridinylsulfonyl, and the like.

“Treating” or “treatment” of a disease includes:
(1) preventing the disease, i.e., causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” may vary depending on the compound, the disease and its severity and the age, weight, etc., of the mammal to be treated.

### Embodiments

(A) In one embodiment, a compound having Formula (I) as defined in the Summary of the Invention is provided.

(B) In one embodiment, a compound having Formula (I) is provided wherein R⁵, R⁶, R¹³, R¹⁴, R¹⁵, R²³ and R²⁴ are as defined below, and the other groups are as defined in the Summary of the Invention:
R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl, wherein the aromatic or alicyclic ring in R⁵ and R⁶ is optionally substituted with one to three substitutents independently selected from R^{a}, R^{b}, and R^{c} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are independently hydrogen or fluoro;
R¹³ is hydrogen, cycloalkyl, cycloalkylalkyl, aryl, heteroaryl, heterocyclyl, aralkyl, heteroaralkyl, heterocyclylalkyl, or —XR¹⁶ (where X is —O—, —CO—, —C(O)O—, —NR¹⁷CO—, —CONR¹⁸—, —NR¹⁹—, —S—, —SO—, —SO₂—, —NR²⁰SO₂—, or —SO₂NR²¹— where R¹⁷, R¹⁸, R¹⁹, R²⁰ and R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl);
R¹⁴ and R¹⁵ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl, wherein the aromatic or alicyclic ring in R¹³, R¹⁴, R¹⁵, and R¹⁶ is optionally substituted with one to three substitutents independently selected from R^{f}, R^{g}, and R^{h} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁱ and R^{j} where Rⁱ and R^{j} are independently hydrogen or fluoro; and
R²³ and R²⁴ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl, wherein the aromatic or alicyclic ring in R²³ and R²⁴ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are independently hydrogen or fluoro.

(1) Within some embodiments of (A) and (B) above, R¹ and R² are alkyl. In certain embodiments, R¹ and R² are methyl.

(2) Within some embodiments of (A) and (B) above, R¹ and R² are haloalkyl. In certain embodiments, R¹ and R² are independently trifluoromethyl or difluoromethyl.

(3) Within some embodiments of (A) and (B) above, R¹ is ethyl, or n- or iso-propyl and R² is methyl.

(i) Within the above embodiments (1), (2), and (3), one group of compounds of Formula (I) is that wherein R³ is a ring of formula (a): 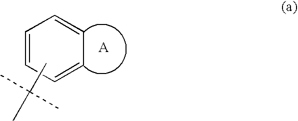
where A is a monocyclic five-, six-, or seven membered heterocyclyl ring substituted with R⁴, R⁵ and R⁶ as defined in the Summary of the Invention.

(ii) Within the above embodiments (1), (2), and (3), another group of compounds of Formula (I) is that wherein R³ is a ring of formula:

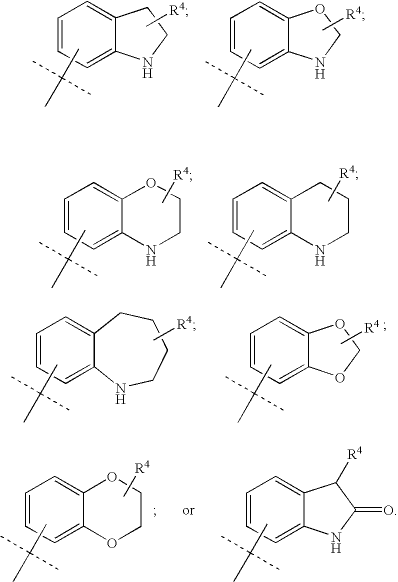

(iii) Within the above embodiments (1), (2), and (3), another group of compounds of Formula (I) is that wherein R³ is a ring of formula:

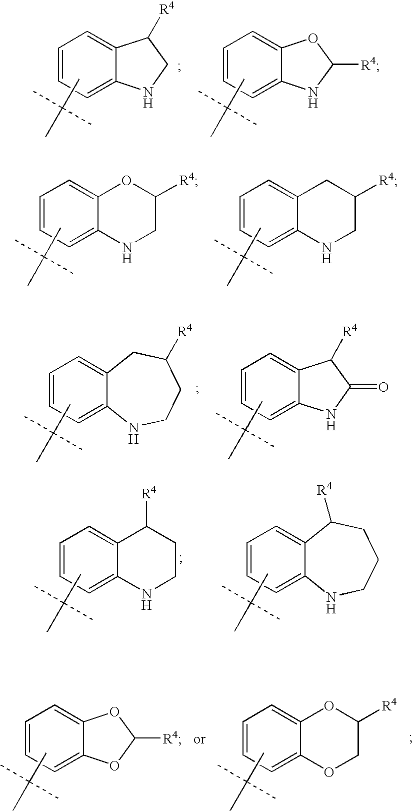

(iv) Within the above embodiments (1), (2), and (3), another group of compounds of Formula (I) is that wherein R³ is a ring of formula:

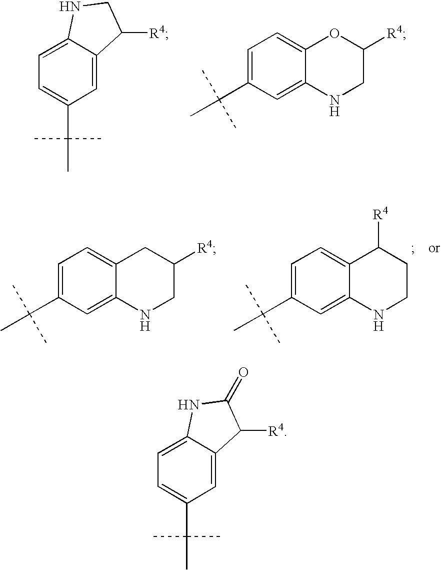

The R⁴ group in groups (ii)-(iv) above, is as defined in the Summary of the invention.

Within the groups (ii)-(iv) above, one group of compounds is that wherein R⁴ is phenyl optionally substituted as defined in the Summary of the Invention.

Within the groups (ii)-(iv) above, another group of compounds is that wherein R⁴ is heteroaryl optionally substituted as defined in the Summary of the Invention.

Within the groups (ii)-(iv) above, another group of compounds is that wherein R⁴ is a saturated monocyclic heterocyclyl optionally substituted as defined in the Summary of the Invention.

Within the groups (ii)-(iv) above, another group of compounds is that wherein R⁴ is saturated fused heterocyclyl optionally substituted as defined in the Summary of the Invention.

The R³ rings in groups (ii)-(iv) above, the subgroups contained therein, including the hydrogen in —NH— groups in the rings, can also be substituted with R⁵ and R⁶ where R⁵ and R⁶ are as defined in the Summary of the Invention or as defined in embodiment (B) above. Within this embodiment, in one group of compounds, one of R⁵ and R⁶ is hydrogen. In another group of compounds, the —NH— groups in the rings are substituted with alkyl, cycloalkyl, or cycloalkylalkyl. In one group of compounds, the —NH— groups in the rings are unsubstituted.

(v) Within the above embodiments (1), (2), and (3), another group of compounds of Formula (I) is that wherein R³ is a ring of formula: 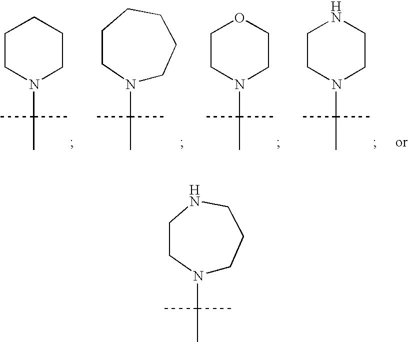
substituted with R²², R²³ and R²⁴ as defined in the Summary of the Invention or as defined in embodiment (B) above, including the hydrogen in —NH— groups in the rings. In one group of compounds, the —NH— groups in the rings are substituted with alkyl, cycloalkyl, or cycloalkylalkyl. In one group of compounds, the —NH— groups in the rings are unsubstituted.

(vi) Within the above embodiments (1), (2), and (3), another group of compounds of Formula (I) is that wherein R³ is a ring of formula: 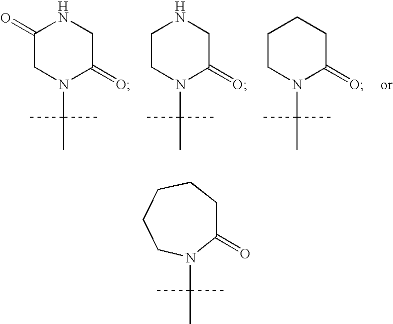
substituted with R²², R²³ and R²⁴ as defined in the Summary of the Invention or as defined in embodiment (B) above, including the hydrogen in —NH— groups in the rings. In one group of compounds, the —NH— groups in the rings are substituted with alkyl, cycloalkyl, or cycloalkylalkyl. In one group of compounds, the —NH— groups in the rings are unsubstituted.

(vii) Within the above embodiments (1), (2), and (3), another group of compounds of Formula (I) is that wherein R³ is a ring of formula: 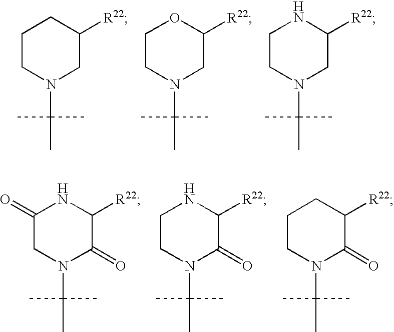
where R²² is as defined in the Summary of the Invention and the rings can also be substituted, including the hydrogen atom on the —NH— group within the ring with R²³ and R²⁴ where R²³ and R²⁴ are as defined in the Summary of the Invention or as defined in embodiment (B) above. Within this embodiment, one group of compounds is that wherein R²³ is hydrogen and R²⁴ is attached to the carbon adjacent to the nitrogen attached to the cinnoline ring. Within this embodiment, one group of compounds is that wherein R²⁴ is hydrogen and R²² is phenyl optionally substituted with R^{k}, R^{l}, and R^{m} are as defined in the Summary of the Invention. Within this embodiment, another group of compounds is that wherein R²⁴ is hydrogen and R²² is saturated heterocyclyl optionally substituted with R^{k}, R^{l}, and R^{m} are as defined in the Summary of the Invention. Within this embodiment, yet another group of compounds is that wherein R²⁴ is hydrogen and R²² is saturated six membered heterocyclyl containing a —C═O group and optionally substituted with R^{k}, R^{l}, and R^{m} are as defined in the Summary of the Invention. Within this embodiment, yet another group of compounds is that wherein R²⁴ is hydrogen and R²² is morpholin-4-yl or 2-oxopiperidin-1-yl optionally substituted with R^{k}, R^{l}, and R^{m} are as defined in the Summary of the Invention.

(viii) Within the above embodiments (1), (2), and (3), another group of compounds of Formula (I) is that wherein R³ is a ring of formula: 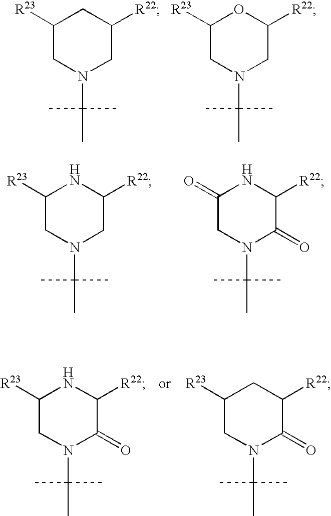
where R²² is phenyl or heteroaryl, each substituted at the para position with R^{k} and optionally substituted with R^{l} and R^{m} wherein R^{k}, R^{l}, and R^{m} are as defined in the Summary of the Invention and R²³ is as defined in the Summary of the Invention or as defined in embodiment (B) above. The —NH— groups in the above rings can optionally be substituted with R²⁴ as defined in the Summary of the Invention or as defined in embodiment (B) above. Within this embodiment, one group of compounds is that wherein R²⁴ is cycloalkyl, alkyl, or cycloalkylalkyl.

(ix) Within the above embodiments (1), (2), and (3), another group of compounds of Formula (I) is that wherein R³ is a ring of formula: 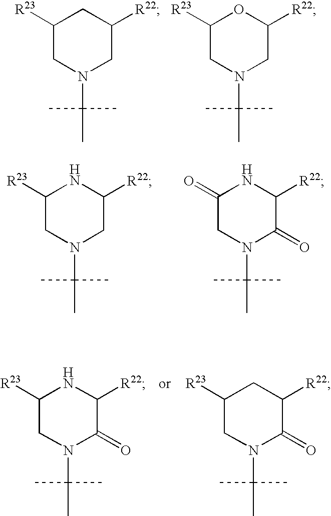
where R²² is heterocyclyl and R²³ is as defined in the Summary of the Invention or as defined in embodiment (B) above. Within this embodiment, one group of compounds is that wherein R²² is heterocyclyl containing at least a —C═O group wherein the heterocyclyl ring is optionally substituted at the para position with R^{k} and optionally substituted with R^{l} and R^{m} wherein R^{k}, R^{l}, and R^{m} are as defined in the Summary of the Invention. Within this embodiment, another group of compounds is that wherein R²² is monocyclic saturated six membered ring containing at least a —C═O group and optionally substituted at the para position with R^{k} and optionally substituted with R^{l} and R^{m} wherein R^{k}, R^{l}, and R^{m} are as defined in the Summary of the Invention. The —NH— groups in the above rings can optionally be substituted with R²⁴ as defined in the Summary of the Invention. Within this embodiment, one group of compounds is that wherein R²⁴ is cycloalkyl, alkyl, or cycloalkylalkyl. Within this embodiment, yet another group of compounds is that wherein R²² is —NHCOR²⁵ where R²⁵ is aryl or heteroaryl as defined in the Summary of the Invention.

(x) Within the above embodiments (1), (2), and (3), yet another group of compounds of Formula (I) is that wherein R³ is a ring of formula: 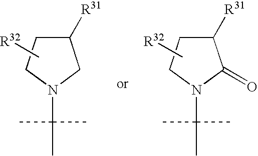
where R³¹ and R³² are as defined in the Summary of the Invention. Within this embodiment one group of compounds is that wherein R³¹ is aryl optionally substituted as defined in the Summary of the Invention.

(xi) Within the above embodiments (1), (2), and (3), another group of compounds of Formula (I) is that wherein R³ is a ring of formula (b). Within this embodiment, one group of compounds is that wherein R³ is a ring of formula: 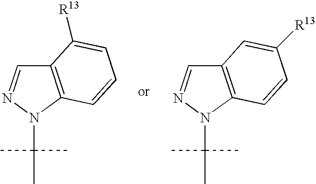
where R¹³ is aryl, heteroaryl, heterocyclyl, aralkyl, heteroaralkyl, heterocyclylalkyl, or —XR¹⁶ (where X is —O—, —CO—, —NR¹⁷CO—, —CONR¹⁸—, —NR¹⁹—, —S—, —SO—, —SO₂—, —NR²⁰SO₂—, or —SO₂NR²¹— where R¹⁷—R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl). Within this embodiment, one group of compounds is that wherein R¹³ is phenyl, heteroaryl or heterocyclyl; and R³ can also be substituted with R¹⁴ and R¹⁵ where R¹⁴ and R¹⁵ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; and wherein the aromatic or alicyclic ring in R¹³, R¹⁴, R¹⁵, and R¹⁶ is optionally substituted with one to three substitutents independently selected from R^{f}, R^{g}, and R^{h} which are independently alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino; and additionally substituted with one or two substitutents independently selected from Rⁱ and R^{j} where Rⁱ and R^{j} are independently hydrogen or fluoro.

Within embodiment (xi), one group of compounds is that wherein R³ is: 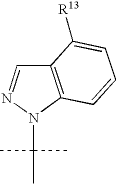
where R¹³ is phenyl, heteroaryl or five or six membered heterocyclyl. Within this embodiment, one group of compounds is that wherein R¹³ is morpholin-4-yl, piperazin-1-yl, or pyridinyl optionally substituted with one to three substitutents independently selected from R^{f}, R^{g}, and R^{h} as defined in the Summary of the Invention.

(xii) Within the above embodiments (1), (2), and (3), another group of compounds of Formula (I) is that wherein R³ is a ring of formula ring of formula (c).

(xiii) Within the above embodiments (1), (2), and (3), another group of compounds of Formula (I) is that wherein R³ is a ring of formula: 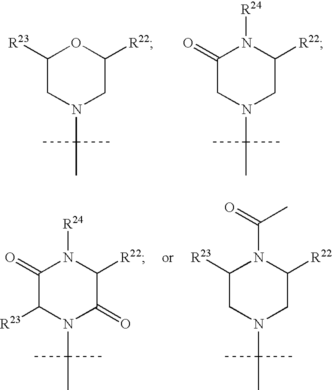
where R²² is phenyl, heteroaryl, or monocyclic saturated five or six membered heterocyclyl ring; R²³ is hydrogen, alkyl, phenyl, heteroaryl, or monocyclic five or six membered heterocyclyl ring; and R²⁴ is alkyl and wherein the aromatic or alicyclic ring in R²² and R²³ is optionally substituted with R^{k}, R^{l} and R^{m} as defined in the Summary of the Invention. Within this embodiment, one group of compounds is that wherein R²⁴ is methyl. Within this subgroup, in one embodiment, R²² is phenyl, heteroaryl, or monocyclic five or six membered heterocyclyl ring and R²³ is hydrogen or alkyl. In another embodiment, R²² and R²³ are independently phenyl, heteroaryl, or monocyclic saturated five or six membered heterocyclyl ring. In each of the above embodiments, the aromatic or alicyclic ring is optionally substituted with R^{k} selected from alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and optionally substituted with R^{l} and R^{m} independently selected from alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, and disubstituted amino.

(xiv) Within the above embodiments (1), (2), and (3), yet another group of compounds of Formula (I) is that wherein R³ is a ring of formula: 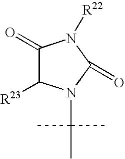
where R²² is aralkyl, preferably benzyl optionally substituted with R^{k}, R^{l} and R^{m} as defined in the Summary of the Invention and R²³ is as defined in the Summary of the Invention. Within this embodiment, a group of compounds is that wherein R²³ is hydrogen or alkyl.

(xv) Within the above embodiments (1), (2), and (3), yet another group of compounds of Formula (I) is that wherein R³ is a ring of formula (a): 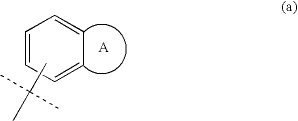
where A is a monocyclic five-, six-, or seven membered heterocyclyl ring and the ring (a) is substituted with R⁴, R⁵ and R⁶ as defined below.

R⁴ is cycloalkyl, cycloalkylalkyl, aryl, heteroaryl, heterocyclyl, aralkyl, heteroaralkyl, heterocyclylalkyl, or —XR⁷ (where X is —O—, —CO—, —NR⁸CO—, —CONR⁹—, —NR¹⁰—, —S—, —SO—, —SO₂—, —NR¹¹SO₂—, or —SO₂NR¹²— where R⁸—R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl).

R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino. Within this embodiment, a group of compounds is that wherein R⁶ is hydrogen.

Within group (xv), the aromatic or alicyclic ring in R⁴, R⁵, R⁶, and R⁷ is optionally substituted with one to three substitutents independently selected from R^{a}, R^{b}, and R^{c} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, and optionally substituted heterocyclyl; and additionally substituted with one or two substitutents independently selected from R^{d} and R^{e} where R^{d} and R^{c} are independently hydrogen or fluoro. In one embodiment, ring A is a saturated five or six membered heterocyclyl ring.

(xvi) Within the above embodiments (1), (2), and (3), yet another group of compounds of Formula (I) is that wherein R³ is a ring of formula (b): 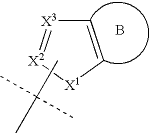
where X¹, X², and X³ are independently carbon, nitrogen, oxygen or sulfur provided that at least two of X¹, X², and are other than carbon; and B is phenyl, or a six-membered heteroaryl ring (wherein the six-membered heteroaryl ring contains one or two nitrogen atoms, the rest of the ring atoms being carbon), or a monocyclic five-, six-, or seven-membered heterocyclyl ring; and wherein ring (b) is substituted with R¹³, R¹⁴ and R¹⁵ as defined below.

R¹³ is cycloalkyl, cycloalkylalkyl, aryl, heteroaryl, heterocyclyl, aralkyl, heteroaralkyl, heterocyclylalkyl, or —XR¹⁶ (where X is —O—, —CO—, —NR¹⁷CO—, —CONR¹⁸—, —NR¹⁹—, —S—, —SO—, —SO₂—, —NR²⁰SO₂—, or —SO₂NR²¹— where R¹⁷—R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl).

R¹⁴ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R¹⁵ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; within this embodiment, a group of compounds is that wherein R¹⁵ is hydrogen.

Within group (xvi), the aromatic or alicyclic ring in R¹³, R¹⁴, R¹⁵, and R¹⁶ is optionally substituted with one to three substitutents independently selected from R^{f}, R^{g}, and R^{h} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁱ and R^{j} where Rⁱ and R^{j} are independently hydrogen or fluoro.

(xvii) Within the above embodiments (1), (2), and (3), yet another group of compounds of Formula (I) is that wherein R³ is a monocyclic six- or seven-membered heterocyclyl ring substituted with R²², R²³ and R²⁴ as defined below.

R²² is aryl, heteroaryl, heterocyclyl, aralkyl, heteroaralkyl, heterocyclylalkyl, or —XR²⁵ (where X is —O—, —CO—, —NR²⁶CO—, —CONR²⁷—, —NR²⁸—, —S—, —SO—, —SO₂—, —NR²⁹SO₂—, or —SO₂NR³⁰— where R²⁶—R³⁰ are independently hydrogen, alkyl, hydroxyalkyl, alkoxyalkyl, aryl, aralkyl, heteroaryl, heteroaralkyl, acyl, or heterocyclylalkyl and R²⁵ is cycloalkyl, cycloalkylalkyl, aryl, heteroaryl, heterocyclyl, aralkyl, heteroaralkyl, or heterocyclylalkyl).

R²³ is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R²⁴ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; within this embodiment, a group of compounds is that wherein R²⁴ is hydrogen.

Within group (xvii), the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are independently hydrogen or fluoro.

(xviii) Within the above embodiments (1), (2), and (3), yet another group of compounds of Formula (I) is that wherein R³ is pyrrolidin-1-yl substituted with R³¹, R³² and R³³ as defined below.

R³¹ is aryl, heteroaryl, heterocyclyl, aralkyl, heteroaralkyl, heterocyclylalkyl, or —XR³⁴ (where X is —O—, —CO—, —NR³⁵CO—, —CONR³⁶—, —NR³⁷—, —S—, —SO—, —SO₂—, —NR³⁸SO₂—, or —SO₂NR³⁹— where R³⁵—R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl).

R³² is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, aryl, heteroaryl or heterocyclyl.

R³³ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino; within this embodiment, a group of compounds is that wherein R³³ is hydrogen.

Within group (xviii), the aromatic or alicyclic ring in R³¹, R³², R³³, and R³⁴ is optionally substituted with one to three substitutents independently selected from R^{p}, R^{q}, and R^{r} which are independently alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, sulfonyl, aminocarbonyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{s} and R^{t} where R^{s} and R^{t} are independently hydrogen or fluoro.

(xix) Within the above embodiments (1), (2), and (3), yet another group of compounds of Formula (I) is that wherein R³ is 2-oxopyrrolidinyl or 2,4-dioxoimidazolidinyl substituted with R³¹, R³² and R³³ as defined below.

R³¹ is aryl, heteroaryl, heterocyclyl, aralkyl, heteroaralkyl, heterocyclylalkyl, or —XR³⁴ (where X is —O—, —CO—, —NR³⁵CO—, —CONR³⁶—, —NR³⁷—, —S—, —SO—, —SO₂—, —NR³⁸SO₂—, or —SO₂NR³⁹— where R³⁵—R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl).

R³² is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, aryl, heteroaryl or heterocyclyl.

R³³ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino; within this embodiment, a group of compound is that wherein R³³ is hydrogen.

Within group (xix), the aromatic or alicyclic ring in R³¹, R³², R³³, and R³⁴ is optionally substituted with one to three substitutents independently selected from R^{p}, R^{q}, and R^{r} which are independently alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, sulfonyl, aminocarbonyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{s} and R^{t} where R^{s} and R^{t} are independently hydrogen or fluoro.

(xx) Within the above embodiments (1), (2), and (3), another group of compounds of Formula (I) is that wherein R³ is a ring of formula: 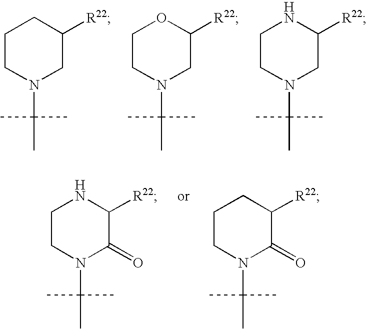
where R²² is phenyl or heteroaryl and the R²² rings are optionally substituted, including the hydrogen atom on the —NH— group within the ring with R²⁴ where R²⁴ are as defined in the Summary of the Invention or as defined in embodiment (B) above. Within this embodiment, one group of compounds is that wherein R²⁴ is hydrogen and R²² is phenyl optionally substituted with R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, and optionally substituted heterocyclyl; and additionally substituted with one or two substitutents independently selected from Rⁿ and R^{o} where Rⁿ and R^{o} are independently hydrogen or fluoro. Within this embodiment, one group of compounds is that wherein R²⁴ is hydrogen and R²² is phenyl substituted with R^{k} as defined above. Within this embodiment, another group of compounds is that wherein R²⁴ is hydrogen and R²² is phenyl substituted with R^{k} and R^{l} as defined above and are located at the 2,6-positions of the phenyl ring, the carbon atom of the phenyl ring attached to the R³ rings shown in (xx) above being the one position. Within this embodiment, yet another group of compounds is that wherein R²⁴ is hydrogen and R²² is phenyl substituted with R^{k} and R^{l} as defined above and are located at the 3,5-positions of the phenyl ring, the carbon atom of the phenyl ring attached to the R³ rings shown in (xx) above being the one position. Within this embodiment, yet another group of compounds is that wherein R²⁴ is hydrogen and R²² is phenyl substituted with R^{k} and R^{l} as defined above and R^{k} and R^{l} are located at the 2,4-positions of the phenyl ring, the carbon atom of the phenyl ring attached to the R³ rings shown in (xx) above being the one position.

(xxi) Within the above embodiments (1), (2), and (3), another group of compounds of Formula (I) is that wherein R¹ and R² are alkyl and R³ is a ring of formula (b) substituted with R¹³ where R¹³ is hydrogen, heteroaryl, heterocyclyl or —XR¹⁶ (where X is —O—, —CONH—, or —NR¹⁹— where R¹⁹ is hydrogen or alkyl and R¹⁶ is cycloalkyl or aralkyl); and R¹⁴ where R¹⁴ is hydrogen or alkoxyalkyloxy wherein the aromatic or alicyclic ring in R¹³ and R¹⁶ is optionally substituted with one to three substitutents independently selected from R^{f}, R^{g}, and R^{h} which are independently alkyl or cyloalkylalkyl provided that one of R¹³ and R¹⁴ is not hydrogen. Within this embodiment, one group of compounds is that wherein R¹ and R² are methyl and R³ is 1H-indazolyl substituted with R¹³ where R¹³ is hydrogen, heteroaryl, heterocyclyl or —XR¹⁶ (where X is —O—, —CONH—, or —NR¹⁹— where R¹⁹ is hydrogen or alkyl and R¹⁶ is cycloalkyl or aralkyl); and R¹⁴ where R¹⁴ is hydrogen or alkoxyalkyloxy wherein the aromatic or alicyclic ring in R¹³ and R¹⁶ is optionally substituted with one to three substitutents independently selected from R^{f}, R^{g}, and R^{h} which are independently alkyl and cyloalkylalkyl provided that one of R¹³ and R¹⁴ is not hydrogen. Within this embodiment, one group of compounds is that wherein R¹ and R² are methyl and R³ is 3-cyclopropylaminocarbonyl-1H-indazol-1-yl; 5-benzyloxy-1H-indazol-1-yl; 6-benzyloxy-1H-indazol-1-yl; 4-(2-methoxyethyloxy)-1H-indazol-1-yl; 5-(2-methoxyethyloxy)-1H-indazol-1-yl; 6-(2-methoxyethyloxy)-1H-indazol-1-yl; 5-(morpholin-4-yl)-1H-indazol-1-yl; 6-(morpholin-4-yl)-1H-indazol-1-yl; 5-(pyridin-3-yl)-1H-indazol-1-yl; 5-(pyridin-4-yl)-1H-indazol-1-yl; 4-(pyridin-4-yl)-1H-indazol-1-yl; 4-(morpholin-4-yl)-1H-indazol-1-yl; 4-(4-methylpiperazin-1-yl)-1H-indazol-1-yl; 4-(piperazin-1-yl)1-1H-indazol-1-yl; 4-(1-ethylpiperazin-4-yl)1-1H-indazol-1-yl; 4-(1-methyl-2-oxo-piperazin-4-yl)1-1H-indazol-1-yl; 4-(1-cyclopropylmethylpiperazin-4-yl)1-1H-indazol-1-yl; 4-pyrrolidin-1-yl-1H-indazol-1-yl; 4-(1-ethylpiperazin-4-yl)-1H-indazol-1-yl.

(xxii) Within the above embodiments (1), (2), and (3), another group of compounds of Formula (I) is that wherein R¹ and R² are alkyl and R is monocyclic six- or seven-membered heterocyclyl ring substituted with R²² where R²² is aryl, heteroaryl, heterocyclyl, aralkyl, heterocyclylalkyl, or —XR²⁵ (where X is —O—, —CO—, —NH⁶CO—, or —NH— where R²⁵ is aryl, heterocyclyl, or aralkyl); and R²³ where R²³ is hydrogen, alkyl, hydroxyl, or acyl; and wherein the aromatic or alicyclic ring in R²², R²³, and R²⁵ is optionally substituted with one to three substitutents independently selected from R^{k}, R^{l}, and R^{m} which are independently alkyl, alkoxy, halo, haloalkoxy, hydroxyl, cyano, and disubstituted amino; or R³ is pyrrolidin-1-yl substituted with R³¹ where R³¹ is aryl, aralkyl, or —XR³⁴ (where X is —NHCO—, or —NH— where R³⁴ is aryl or aralkyl wherein the aromatic ring in R³¹ is optionally substituted with one to three substitutents independently selected from R^{p}, R^{q}, and R^{r} which are alkoxy.

Within this embodiment, one group of compounds is that wherein R¹ and R² are methyl and R³ is 2-(RS)-phenylmorpholin-4-yl; 2-(R)-phenylmorpholin-4-yl; 2-(S)-phenylmorpholin-4-yl; 2-(RS)-(4-methoxyphenyl)morpholin-4-yl; 3-(RS)-phenylpyrrolidin-1-yl; 2-(RS)-(4-fluorophenyl)morpholin-4-yl; 2-(RS)-(2-chlorophenyl)morpholin-4-yl; 2-(RS)-(pyridin-3-yl)morpholin-4-yl; 4-(RS)-(phenoxy)piperidin-1-yl; 2-(RS)-(pyrrolidin-1-ylmethyl)-morpholin-4-yl; 3-(RS)-(2-oxopiperidin-1-yl)piperidin-1-yl; 2-(RS)-(benzyl)pyrrolidin-1-yl; 4-methyl-3-(RS)-(phenyl)piperazin-1-yl; 3-(RS)-(pyrrolidin-1-ylcarbonyl)piperidin-1-yl; 3-(RS)-benzylpiperidin-1-yl; 3-(R)-(2-oxopiperidin-1-yl)piperidin-1-yl; 3-(S)-(2-oxopiperidin-1-yl)piperidin-1-yl; 3-(RS)-indol-1-ylpiperidin-1-yl; 3-(RS)-(phenoxy)piperidin-1-yl; 2-(RS)-(2,3-dihydrobenzofuran-5-yl)morpholin-4-yl; 3-(RS)-(piperidin-1-ylcarbonyl)piperidin-1-yl; 2-(RS)-(4-chlorophenyl)-5-oxo-morpholin-4-yl; 2-(S)-(4-methoxyphenyl)morpholin-4-yl; 2-(R)-(4-methoxyphenyl)morpholin-4-yl; 3-(RS)-(2-fluorophenyl)piperidin-1-yl; 3-(RS)-(phenyl)piperazin-1-yl; 1-acetyl-3-(RS)-(phenyl)piperazin-1-yl; 2-(RS)-(4-fluorophenyl)-2-methylmorpholin-4-yl; 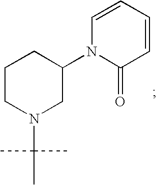
2-(RS)-(3-methoxyphenyl)-3-oxomorpholin-4-yl; 2-(RS)-(3-methoxyphenyl)morpholin-4-yl; 2-(RS)-(2-methoxyphenyl)piperidin-1-yl; 3-(RS)-(3-methoxyphenyl)piperazin-1-yl; 3-(RS)-(3-methyl-[1.2.4]oxadiazol-5-yl)piperidin-1-yl; 2-(RS)-(4-chlorophenyl)morpholin-4-yl; 2-(RS)-(4-methylphenyl)morpholin-4-yl; 2-(RS)-(3,5-dichlorophenyl)morpholin-4-yl; 2-(RS)-(3-iodo-4-methoxyphenyl)morpholin-4-yl; 6-methyl-2-(RS)-(4-methoxyphenyl)morpholin-4-yl; 3-(RS)-(2-oxopyrrolidin-1-ylmethyl)piperidin-1-yl; 2-(RS)-(4-trifluoromethoxyphenyl)morpholin-4-yl; 3-hydroxy-3-(4-methoxyphenyl)piperidin-1-yl; 6-oxo-2-(RS)-(4-methoxyphenyl)-1-methylpiperazin-4-yl; 5-(4-methoxyphenyl)-1,2,3,4-tetrahydropyridin-1-yl; 3-(S)-(2-fluorophenyl)piperidin-1-yl; 3-(R)-(2-fluorophenyl)piperidin-1-yl; 2-(RS)-(4-methoxyphenyl)-1-methylpiperazin-4-yl; 1-acetyl-3-(RS)-(phenyl)piperazin-1-yl; 2-(RS)-(4-hydroxyphenyl)morpholin-4-yl; 3-(RS)-(4-methoxybenzylamino)pyrrolidin-1-yl; 2-(RS)-(4-methoxyphenylamino)-3-methylmorpholin-4-yl; 4-(RS)-(4-methoxybenzylamino)piperidin-1-yl; 3-(RS)-(3,5-dimethoxyphenyl)piperazin-4-yl; 3-(RS)-1-(4-methoxyphenyl)piperidin-3-yl; 3-(RS)-(2-oxopyrrolidin-1-yl)piperidin-1-yl; 4-(RS)-(2-oxopiperidin-1-yl)piperidin-1-yl; 4-(RS)-(2-oxoazetidin-1-yl)piperidin-1-yl; 4-(RS)-(2-oxopyrrolidin-1-yl)piperidin-1-yl; 2-(RS)-(6-methoxynaphth-2-yl)piperazin-4-yl; 2-(RS)-(4-methoxyphenyl)piperazin-4-yl; 2-(RS)-(2-flouro-4-methylphenyl)morpholin-4-yl; 2-(S)-(2-chlorophenyl)morpholin-4-yl; 2-(R)-(2-chlorophenyl)morpholin-4-yl; 2-(RS)-(2-cyanophenyl)morpholin-4-yl; 2-(RS)-(2,6-diflouorphenyl)morpholin-4-yl; 2-(RS)-(thiophen-2-yl)piperazin-4-yl; 2-(RS)-(4-dimethylaminophenyl)piperazin-4-yl; 3-(RS)-(4-methoxyphenylcarbonylamino)-pyrrolidin-1-yl; 2(RS)-(2-methylphenyl)morpholin-4-yl; 3-(RS)-(naphth-2-yl)piperazin-1-yl; 3-(RS)-(4-methoxybenzyl)pyrrolidin-1-yl; 3-(RS)-(phenylcarbonylamino)piperidin-1-yl; 2-(RS)-ethyl-6-(RS)-(4-methoxyphenyl)morpholin-4-yl; and 1-(4-methoxyphenyl)-1,2,5,6-tetrahydropyridin-3-yl.

Representative compounds of Formula (I) are provided in Table 1 below.

TABLE 1

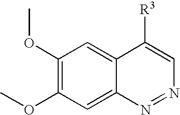

| Cpd # | R³ |
|---|---|
| 1 | benzoxazol-2-yl |
| 2 | indazol-1-yl |
| 3 | 3-cyclopropylaminocarbonyl-1H-indazol-1-yl |
| 4 | 2-(RS)-phenylmorpholin-4-yl |
| 5 | 2,3-dihydrobenzo[b][1,4]dioxin-6-yl |
| 6 | 5-benzyloxy-1H-indazol-1-yl |
| 7 | 6-benzyloxy-1H-indazol-1-yl |
| 8 | 4-methyl-3,4-dihydro-2H-benzo[b][1,4]oxazin-7-yl |
| 9 | 2-(R)-phenylmorpholin-4-yl |
| 10 | 2-(S)-phenylmorpholin-4-yl |
| 11 | 2-(RS)-(4-methoxyphenyl)morpholin-4-yl |
| 12 | 3-(RS)-phenylpyrrolidin-1-yl |
| 13 | 2-(RS)-(4-fluorophenyl)morpholin-4-yl |
| 14 | 2-(RS)-(2-chlorophenyl)morpholin-4-yl |
| 15 | 2-(RS)-(pyridin-3-yl)morpholin-4-yl |
| 16 | 4-(RS)-(phenoxy)piperidin-1-yl |
| 17 | 4-(2-methoxyethyloxy)-1H-indazol-1-yl |
| 18 | 5-(2-methoxyethyloxy)-1H-indazol-1-yl |
| 19 | 6-(2-methoxyethyloxy)-1H-indazol-1-yl |
| 20 | 5-(morpholin-4-yl)-1H-indazol-1-yl |
| 21 | 6-(morpholin-4-yl)-1H-indazol-1-yl |
| 22 | 5-(pyridin-3-yl)-1H-indazol-1-yl |
| 23 | 5-(pyridin-4-yl)-1H-indazol-1-yl |
| 24 | 4-(pyridin-4-yl)-1H-indazol-1-yl |
| 25 | 4-(morpholin-4-yl)-1H-indazol-1-yl |
| 26 | 4-(4-methylpiperazin-1-yl)-1H-indazol-1-yl |
| 27 | 2-(RS)-(pyrrolidin-1-ylmethyl)morpholin-4-yl |
| 28 | 3-(RS)-(2-oxopiperidin-1-yl)piperidin-1-yl |
| 29 | 2-(RS)-(benzyl)pyrrolidin-1-yl |
| 30 | 4-methyl-3-(RS)-(phenyl)piperazin-1-yl |
| 31 | 1,3-benzodioxol-5-yl |
| 32 | 3-(RS)-(pyrrolidin-1-ylcarbonyl)piperidin-1-yl |
| 33 | 3-(RS)-benzylpiperidin-1-yl |
| 34 | 3-(R)-(2-oxopiperidin-1-yl)piperidin-1-yl |
| 35 | 3-(S)-(2-oxopiperidin-1-yl)piperidin-1-yl |
| 36 | 3-(RS)-indol-1-ylpiperidin-1-yl |
| 37 | 3-(RS)-(phenoxy)piperidin-1-yl |
| 38 | 2-(RS)-(2,3-dihydrobenzofuran-5-yl)morpholin-4-yl |
| 39 | 3-(RS)-(piperidin-1-ylcarbonyl)piperidin-1-yl |
| 40 | 2-(RS)-(4-chlorophenyl)-5-oxo-morpholin-4-yl |
| 41 | 2-(S)-(4-methoxyphenyl)morpholin-4-yl |
| 42 | 2-(R)-(4-methoxyphenyl)morpholin-4-yl |
| 43 | 3-(RS)-(2-fluorophenyl)piperidin-1-yl |
| 44 | 3-(RS)-(phenyl)piperazin-1-yl |
| 45 | 1-acetyl-3-(RS)-(phenyl)piperazin-1-yl |
| 46 | 4-(piperazin-1-yl)1-1H-indazol-1-yl |
| 47 | 2-(RS)-(4-fluorophenyl)-2-methylmorpholin-4-yl |
| 48 | 4-(1-ethylpiperazin-4-yl)1-1H-indazol-1-yl |
| 49 | 4-(1-methyl-2-oxo-piperazin-4-yl)1-1H-indazol-1-yl |
| 50 | 4-(1-cyclopropylmethylpiperazin-4-yl)1-1H-indazol-1-yl |
| 51 | 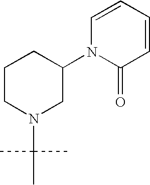 |
| 52 | 2-(RS)-(3-methoxyphenyl)-3-oxomorpholin-4-yl |
| 53 | 2-(RS)-(3-methoxyphenyl)morpholin-4-yl |
| 54 | 2-(RS)-(2-methoxyphenyl)piperidin-1-yl |
| 55 | 3-(RS)-(3-methoxyphenyl)piperazin-1-yl |
| 56 | 3-(RS)-(3-methyl-[1.2.4]oxadiazol-5-yl)piperidin-1-yl |
| 57 | 2-(RS)-(4-chlorophenyl)morpholin-4-yl |
| 58 | 2-(RS)-(4-methylphenyl)morpholin-4-yl |
| 59 | 2-(RS)-(3,5-dichlorophenyl)morpholin-4-yl |
| 60 | 2-(RS)-(3-iodo-4-methoxyphenyl)morpholin-4-yl |
| 61 | 6-methyl-2-(RS)-(4-methoxyphenyl)morpholin-4-yl |
| 62 | 3-(RS)-(2-oxopyrrolidin-1-ylmethyl)piperidin-1-yl |
| 63 | 2-(RS)-(4-trifluoromethoxyphenyl)morpholin-4-yl |
| 64 | 3-hydroxy-3-(4-methoxyphenyl)piperidin-1-yl |
| 65 | 6-oxo-2-(RS)-(4-methoxyphenyl)-1-methylpiperazin-4-yl |
| 66 | 5-(4-methoxyphenyl)-1,2,3,4-tetrahydropyridin-1-yl |
| 67 | 3-(S)-(2-fluorophenyl)piperidin-1-yl |
| 68 | 3-(R)-(2-fluorophenyl)piperidin-1-yl |
| 69 | 2-(RS)-(4-methoxyphenyl)-1-methylpiperazin-4-yl |
| 70 | 1-(methyoxycarbonylmethyl)-3-(RS)- |
| | (3-methoxyphenyl)piperazin-1-yl |
| 71 | 4-dimethylamino-1H-indazol-1-yl |
| 72 | 4-ethylmethylamino-1H-indazol-1-yl |
| 73 | 4-pyrrolidin-1-yl-1H-indazol-1-yl |
| 74 | 2-(RS)-(4-hydroxyphenyl)morpholin-4-yl |
| 75 | 3-(RS)-(4-methoxybenzylamino)pyrrolidin-1-yl |
| 76 | 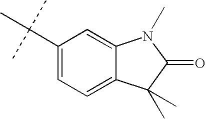 |
| 77 | 2-(RS)-(4-methoxyphenyl)-3-methylmorpholin-4-yl |
| 78 | 4-(RS)-(4-methoxybenzylamino)piperidin-1-yl |
| 79 | 3-(RS)-(3,5-dimethoxyphenyl)piperazin-4-yl |
| 80 | 4-(1-ethylpiperazin-4-yl)-1H-indazol-1-yl |
| 81 | 3-(RS)-1-(4-methoxyphenyl)piperidin-3-yl |
| 82 | 3-(RS)-(2-oxopyrrolidin-1-yl)piperidin-1-yl |
| 83 | 4-(RS)-(2-oxopiperidin-1-yl)piperidin-1-yl |
| 84 | 4-(RS)-(2-oxoazetidin-1-yl)piperidin-1-yl |
| 85 | 4-(RS)-(2-oxopyrrolidin-1-yl)piperidin-1-yl |
| 86 | 2-(RS)-(6-methoxynaphth-2-yl)piperazin-4-yl |
| 87 | 2-(RS)-(4-methoxyphenyl)piperazin-4-yl |
| 88 | 2-(RS)-(2-fluoro-4-methylphenyl)morpholin-4-yl |
| 89 | 2-(S)-(2-chlorophenyl)morpholin-4-yl |
| 90 | 2-(R)-(2-chlorophenyl)morpholin-4-yl |
| 91 | 2-(RS)-(2-cyanophenyl)morpholin-4-yl |
| 92 | 2-(RS)-(2,6-difluorophenyl)morpholin-4-yl |
| 93 | 2-(RS)-(thiophen-2-yl)piperazin-4-yl |
| 94 | 2-(RS)-(4-dimethylaminophenyl)piperazin-4-yl |
| 95 | 3-(RS)-(4-methoxyphenylcarbonylamino)pyrrolidin-1-yl |
| 96 | 2-(RS)-(2-methylphenyl)morpholin-4-yl |
| 97 | 3-(RS)-(naphth-2-yl)piperazin-1-yl |
| 98 | 3-(RS)-(4-methoxybenzyl)pyrrolidin-1-yl |
| 99 | 3-(RS)-(phenylcarbonylamino)piperidin-1-yl |
| 100 | 2-(RS)-ethyl-6-(RS)-(4-methoxyphenyl)morpholin-4-yl |
| 101 | 1-(4-methoxyphenyl)-1,2,5,6-tetrahydropyridin-3-yl |
| 102 | 3-(RS)-(2-fluorophenylcarbonylamino)piperidin-1-yl |
| 103 | 2-(RS)-(naphth-1-yl)piperazin-4-yl |
| 104 | 3-(RS)-(cyclohexyl)piperidin-1-yl |
| 105 | 4-(RS)-(phenyl)azepan-1-yl |
| 106 | 4-(4-acetylpiperazin-1-yl)-1H-indazol-1-yl |
| 107 | 3-(RS)-(—N(CH₃)COphenyl)piperidin-1-yl |
| 108 | 3-(RS)-(4-chlorophenylcarbonylamino)piperidin-1-yl |
| 109 | 2-(R)-(2-fluoro-4-methylphenyl)morpholin-4-yl |
| 110 | 2-(S)-(2-fluoro-4-methylphenyl)morpholin-4-yl |
| 111 | 2-(R)-(6-methoxynaphth-2-yl)piperazin-4-yl |
| 112 | 2-(S)-(6-methoxynaphth-2-yl)piperazin-4-yl |
| 113 | 4-bromo-1H-indazol-1-yl |
| 114 | 2-(RS)-(2-hydroxypyridin-5-yl)morpholin-4-yl |
| 115 | 2-(RS)-(4-methoxyphenyl)-5(RS)-methylmorpholin-4-yl |
| 116 | 2-(RS)-(3,4-dimethoxyphenyl)morpholin-4-yl |
| 117 | 2-(RS)-(2-fluoro-4-methoxyphenyl)morpholin-4-yl |
| 118 | 2-(RS)-(3,5-dibenzyloxyphenyl)piperazin-4-yl |
| 119 | 2-(S)-(naphth-2-yl)piperazin-4-yl |
| 120 | 2-(R)-(naphth-2-yl)piperazin-4-yl |
| 121 | 3-(RS)-(3,5-dimethoxyphenylcarbonylamino)piperidin-1-yl |

Representative compounds of Formula (I) are provided in Table 2 below.

TABLE 2

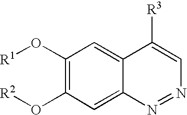

| CPD # | R¹ | R² | R³ |
|---|---|---|---|
| 122 | ethyl | methyl | 2-(RS)-(4-methoxyphenyl)morpholin-4-yl |
| 123 | ethyl | methyl | 2-(R)-(4-methoxyphenyl)morpholin-4-yl |
| 124 | ethyl | methyl | 3-(RS)-(3-oxopiperidin-1-yl)piperidin-1-yl |
| 125 | —CH₂CF₃ | methyl | 2-(RS)-(4-methoxyphenyl)morpholin-4-yl |
| 126 | n-propyl | methyl | 2-(RS)-(4-methoxyphenyl)morpholin-4-yl |
| 127 | methyl | H | 2-(RS)-(4-methoxyphenyl)morpholin-4-yl |
| 128 | H | methyl | 2-(RS)-(4-methoxyphenyl)morpholin-4-yl |

General Synthetic Schemes

Compounds of this invention can be made by the methods depicted in the reaction schemes shown below.

The starting materials and reagents used in preparing these compounds are either available from commercial suppliers such as Aldrich Chemical Co., (Milwaukee, Wis.) Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmoshperic pressure over a temperature range from about −78° C. to about 150° C., such as from about 0° C. to about 125° C., for example, at about room (or ambient) temperature, e.g., about 20° C.

Compounds of Formula (I) where R¹, R² and R³ are as defined in the Summary of the Invention can be prepared as described in Scheme 1 below.

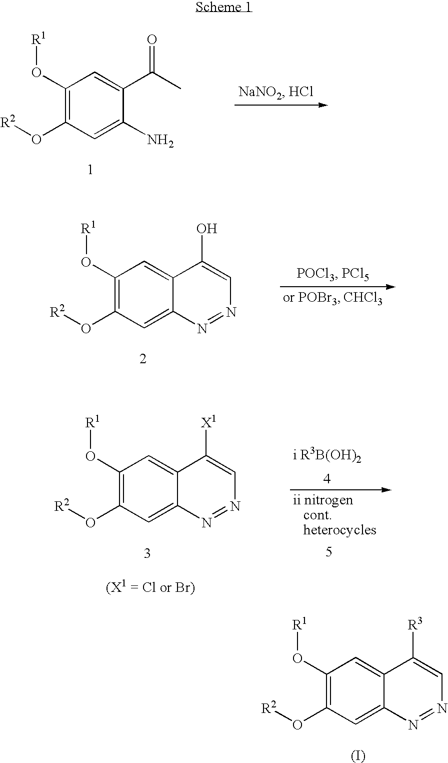

Treatment of 2-amino-4,5-dialkoxyacetophenones 1 with sodium nitrite in concentrated HCl and water provides diazo compound intermediates that cyclize upon heating to provide 6,7-dialkoxy-4-hydroxycinnolines 2. Treatment of 2 with either phosphorous oxychloride or phosphorous oxybromide provides the corresponding chloro or bromo compound of formula 3. The chloro derivative is prepared by heating 2 in neat phosphorous oxychloride, followed by recrystallization of the product after neutralization (see Castle et al., J. Org. Chem. 17:1571, 1952). The bromo derivative is prepared by mixing a concentrated suspension of the 4-hydroxycinnoline in chloroform and phosphorous oxybromide at room temperature and then warming to reflux for 8 to 16 h. Extractive workup after neutralization and subsequent recrystallization from alcoholic solvent such as ethanol provides 4-bromocinnoline.

Compounds of formula 1 are either commercially available (e.g., 2-amino-4,5-dimethoxyacetophenone) or can be synthesized by methods well known in the art. For example, simple dialkyl ethers, wherein the alkyl groups at the 3,4-postions are the same, can be readily prepared under standard etherification reaction conditions. For example, 3,4-dihydroxy-acetophenone can be treated with an excess of a base such as cesium carbonate and the desired alkyl halide to directly provide the dialkylated product. Other bases such as triethylamine, sodium hydride, potassium carbonate, potassium hydride, etc. can be employed in combination with a variety of solvents such as acetone, acetonitrile, DMF, and THF, and the like. 2-Amino-4,5-dialkoxyacetophenones 1 are prepared by nitration with nitric acid in one of several solvents including acetic acid or sulfuric acid at ice bath temperatures to provide 2-nitro-4,5-dialkoxyacetophenones (Iwamura et al., Bioorg. Med. Chem. 10:675, 2002). Reduction of the nitro group under known reaction conditions e.g., hydrogenation with palladium on carbon, iron powder in acetic acid, or nickel boride, among others, provides the desired compounds 1. (Castle et al., J. Org. Chem. 19:1117 1954).

Compounds of formula 1 where R¹ and R² are different can also be prepared by methods well known in the art. For example if the desired substituent at the 3-position is the methyl ether, acetovanillone (3-methoxy-4-hydroxyacetophenone) can be utilized as a starting material. Simple etherification, as described above, can be utilized to provide the required 4-substitution, followed by nitration and reduction steps as described above. Alternatively, compounds of formula 1 can be prepared under Mitsunobu reaction conditions by treating phenol with diethyl or diisopropyl azo-dicarboxylates, triphenylphosphine, and the desired alkyl alcohol in THF solution to give the corresponding alkoxy derivative. Treatment of the phenol with haloacetic acid e.g., chlorodifluoroacetic acid under basic conditions provides difluoromethyl ether.

If compounds of formula 1 where R¹ is other than methyl are desired, 3,4-dihydroxyacetophenone can be utilized as the starting material. 3,4-Dihydroxyacetophenone can be selectively protected as its 4-benzyl ether (Greenspan et al., J. Med. Chem. 42:164, 1999) by treatment with benzyl bromide and lithium carbonate in DMF solution. Functionalization of the 3-OH group with the desired alkyl halide can be accomplished under the esterification conditions described above, including Mitsunobu reaction. Removal of the benzyl ether by hydrogenolysis with palladium on carbon in alcoholic solvents such as methanol and followed by etherification of the 4-OH yields the 3,4-dialkoxyacetophenones. Nitration of 3,4-dialkoxyacetophenones, followed by reduction of the nitro group provides the desired compound 1.

4-Bromo-6,7-bis-difluoromethoxycinnoline analogs can be prepared from 3,4-dimethoxyacetophenone by reaction with nitric acid to yield 3,4-dimethoxy-6-nitroacetophenone which upon treatment with pyridine-HCl provides 1-(4,5-dihydroxy-2-nitrophenyl)ethanone. Treatment of 1-(4,5-dihydroxy-2-nitrophenyl)ethanone with chlorodifluoroacetic acid provides 1-(4,5-bis(difluoromethoxy)-2-nitrophenyl)ethanone which upon reduction of the nitro group to amino group followed by cyclization under conditions described above provides the desired compound. Compounds of formula 2 can also be prepared from 2-alkynylanilines as described in Queguiner et al., Tetrahedron 56:5499, 2000.

Compound 3 is then converted to a compound of Formula (I) where R³ is a group of formula (a)-(c) by reacting it with aryl or heteroaryl boronic acids under Suzuki coupling reaction conditions.

Compounds of Formula (I) where R³ is heterocyclic ring attached to the cinnoline ring via a nitrogen atom, e.g., pyrrolidin-1-yl, piperidin-1-yl, morpolin-4-yl, and the like, can be prepared by reacting 3 with the heterocyclic ring where X¹ is halo or other suitable leaving group such as tosylate, triflate, mesylate and the like in the presence of a base such as triethylamine, pyridine, and the like. Suitable solvents include, and are not limited to, tetrahydrofuran, DMF, and the like. Alternatively, compounds of Formula (I) can be prepared by heating 3 with the heterocyclic ring in a suitable organic solvent such as THF, benzene, dioxane, toluene, alcohol or mixtures thereof, optionally in the presence of a base.

Compounds of formulae 4 and 5 are either commercially available or they can be prepared by methods well known in the art. For example, 3-hydroxy-5-arylpiperidines can be prepared by the methods disclosed in U.S. Pat. No. 4,387,230, the disclosure of which is incorporated herein by reference in its entirety. 3-Hydroxy-5-arylpiperidines can be converted to hydroxy derivatives such as alkoxy, alkoxyalkyloxy or hydroxyalkoxy under alkylation reaction conditions known in the art. Compounds of Formula (I) wherein R³ is a ring of formula (a), such as those shown in embodiments (i)-(iv) and (xv) above, may be prepared by standard synthetic methods known to one of ordinary skill in the art, for example, by Suzuki type coupling of the corresponding boronic acid with 4-bromo-cinnoline 3. (See, e.g., Miyaura and Suzuki, Chem. Rev. 95:2457-2483, 1995). Such boronic acids are either commercially available (e.g., Aldrich Chemical Co. (Milwaukee, Wis.), Lancaster Synthesis (Ward Hill, Mas.), or Maybridge (Conrwall, UK)) or can readily be prepared from the corresponding bromides by methods described in the literature (see, e.g., Miyaura et al., Tetrahedron Letters 1979, 3437; N. Miyaura, A. Suzuki, Chem. Commun. 1979, 866).

Compounds of Formula (I) wherein R³ is a ring of a formula as shown in, for example, embodiments (v)-(xiv) above (e.g., wherein R³ is an N-substituted pyrrolidine, piperidine, homopiperidine, piperazine, homopiperazine, morpholine and the like) can be prepared by Buchwald coupling of the 4-bromocinnoline 3 with the appropriately substituted heterocyclic compound. Such heterocyclic compounds (pyrrolidines, piperidines, homopiperidines, piperazines, homopiperazines, morpholines and the like) are either commercially available or can be readily prepared by standard methods known within the art (see, for example, J. Louie, J. F. Hartwig, Tetrahedron Letters 36, 3609 (1995); A. S. Guram et al., Angew Chem. Int. Ed. 34, 1348 (1995)).

Substituted indazoles useful to make compounds of Formula (I) wherein R³ is a ring as shown in embodiment (xi) above are either commercially available (e.g., Aldrich Chemical Co., Sinova, Inc. (Bethesda, Ma.), J & W PharmLab, LLC (Morrisville, Pa.)) or can be prepared by methods commonly known within the art (see, for example, Synthesis of 1-Aryl-1H-indazoles via Palladium-Catalyzed Intramolecular Amination of Aryl Halides, Lebedev, A. Y.; Khartulyari, A. S.; Voskoboynikov, A. Z. J. Org Chem. 2005; 70(2); 596-602. and the references cited therein). For example, indazoles wherein R¹³ is heterocyclyl, for example, morpholine or N-methylpiperazine, may be synthesized by Buchwald-type coupling of the corresponding bromoindazole with the desired heterocyclic compound. The bromoindazoles may prepared as described in International Publication No. WO 2004/029050, the disclosure of which is incorporated herein by reference in its entirety. Copper catalyzed reaction of the appropriately substituted indazole with 4-bromocinnoline 3 provides the appropriate compound of Formula (I). Alternatively, the bromoindazole undergoes palladium catalyzed reaction with 4-bromocinnoline 3 to provide 6,7-dimethoxy-4-(bromo-1H-indazol-1-yl)cinnoline. Subsequent N-arylation reaction with, for example morpholine or N-methylpiperazine provides the desired compound of Formula I. Alternatively, Suzuki-type reaction of 6,7-dimethoxy-4-(bromo-1H-indazol-1-yl)cinnoline with aryl or heteroaryl boronic acids, for example, phenylboronic acid or 4-pyridine boronic acid, gives the corresponding aryl or heteroaryl substituted indazole cinnoline of Formula (I).

Utility and Methods of Use

In one aspect, methods are provided for treating a disorder or disease treatable by inhibition of PDE10 comprising administering a therapeutically effective amount of compound as provided herein to a patient in need thereof to treat the disorder or disease.

The compounds of the present invention inhibit PDE10 enzyme activity and hence raise the levels of cAMP or cGMP within cells that express PDE10. Accordingly, inhibition of PDE10 enzyme activity can be useful in the treatment of diseases caused by deficient amounts of cAMP or cGMP in cells. PDE10 inhibitors can be of benefit in cases wherein raising the amount of cAMP or cGMP above normal levels results in a therapeutic effect. Inhibitors of PDE10 can be used to treat disorders of the peripheral and central nervous system, cardiovascular diseases, cancer, gastro-enterological diseases, endocrinological diseases and urological diseases.

Indications that may be treated with PDE10 inhibitors, either alone or in combination with other drugs, include, but are not limited to, those diseases thought to be mediated in part by the basal ganglia, prefrontal cortex and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention can be used in treating patients suffering from all forms of psychoses, including, but not limited to, schizophrenia, late-onset schizophrenia, schizoaffective disorders, prodromal schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways. (Saxena et al., Br. J. Psychiatry Suppl. 35:26-37, 1998). Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The compounds of the present invention can therefore be useful for the indication of OCD. OCD may result, in some cases, from streptococcal infections that cause autoimmune reactions in the basal ganglia (Giedd et al., Am J Psychiatry. 57:281-3, 2000). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that since PDE10 degrades cAMP or cGMP, the level of this enzyme affects memory in animals, for example, in humans. For example, a compound that inhibits cAMP phosphodiesterase (PDE) can thereby increase intracellular levels of cAMP, which in turn activate a protein kinase that phosphorylates a transcription factor (cAMP response binding protein), which transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The compounds of the present invention can be used for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. In certain embodiments, the present invention provides methods for dealing with memory loss separate from dementia, including mild cognitive impairment (MCI) and age-related cognitive decline. In some embodiments, the present invention provides methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention can be used in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The compounds of the present invention invention are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 also called Machado-Joseph disease, MJD (ataxin-3); spinocerebellar ataxia type-6 (alpha 1a-voltage dependent calcium channel); spinocerebellar ataxia type-7 (ataxin-7); and spinal and bulbar muscular atrophy, SBMA, also know as Kennedy disease (androgen receptor).

The basal ganglia are important for regulating the function of motor neurons; disorders of the basal ganglia result in movement disorders. Most prominent among the movement disorders related to basal ganglia function is Parkinson's disease (Obeso J A et al., Neurology., Jan. 13, 2004 ; 62(1 Suppl 1):S17-30). Other movement disorders related to dysfunction of the basla ganglia include tardive dyskinesia, progressive supranuclear palsy and cerebral palsy, corticobasal degeneration, multiple system atrophy, Wilson disease, and dystonia, tics, and chorea. The compounds of the invention can be used to treat movement disorders related to dysfunction of basal ganglia neurons.

PDE10 inhibitors can be used to raise cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the compounds of the invention can be used to stop disease progression or restore damaged circuits in the brain by a combination of effects including increased synaptic plasticity, neurogenesis, anti-inflammatory, nerve cell regeneration and decreased apoptosis

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity may inhibit cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Therefore, compounds disclosed in this invention can be used to stop the growth of cancer cells that express PDE10.

The compounds of the invention can also be suitable for use in the treatment of diabetes and related disorders such as obesity, by focusing on regulation of the cAMP signaling system. By inhibiting PDE-10A activity, intracellular levels of cAMP are increased, thereby increasing the release of insulin-containing secretory granules and, therefore, increasing insulin secretion. See, for example, WO 2005/012485, which is hereby incorporated by reference in its entirety. The compounds of Formula (I) can also be used to treat diseases disclosed in U.S. Patent application publication No. 2006/019975, the disclosure of which is incorporated herein by reference in its entirety.

Testing

The PDE10 inhibitory activities of the compounds of the present invention can be tested, for example, using the in vitro or in vivo assays described in working Examples 21 and 22 below.

Administration and Pharmaceutical Compositions

In general, the compounds provided herein can be administered in a therapeutically effective amount by any of the accepted modes of administration for agents that serve similar utilities. The actual amount of the compound of this invention, i.e., the active ingredient, may depend upon numerous factors such as the severity of the disease to be treated, the age and relative health of the subject, the potency of the compound used, the route and form of administration, and other factors.

Therapeutically effective amounts of compounds of formula (I) may range from approximately 0.1-1000 mg per day; preferably 0.5 to 250 mg/day, more preferably 3.5 mg to 70 mg per day.

In general, compounds of this invention may be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

The compounds can be administered as the sole active agent or in combination with other pharmaceutical agents such as other agents used in the treatment of psychoses, especially schizophrenia and bipolar disorder, obsessive-compulsive disorder, Parkinson's disease, Alzheimer's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range and can be administered either simultaneously or sequentially.

Drugs suitable in combination with the compounds of the present invention include, but not limited to, other suitable schizophrenia drugs such as Clozaril, Zyprexa, Risperidone, and Seroquel; bipolar disorder drugs such as Lithium, Zyprexa, and Depakote, Parkinson's disease drugs such as Levodopa, Parlodel, Permax, Mirapex, Tasmar, Contan, Kemadin, Artane, and Cogentin; agents used in the treatment of Alzheimer's disease such as, but not limited to, Reminyl, Cognex, Aricept, Exelon, Akatinol, Neotropin, Eldepryl, Estrogen and Cliquinol; agents used in the treatment of dementia such as, but not limited to, Thioridazine, Haloperidol, Risperidone, Cognex, Aricept, and Exelon;agents used in the treatment of epilepsy such as, but not limited to, Dilantin, Luminol, Tegretol, Depakote, Depakene, Zarontin, Neurontin, Barbita, Solfeton, and Felbatol; agents used in the treatment of multiple sclerosis such as, but not limited to, Detrol, Ditropan XL, OxyContin, Betaseron, Avonex, Azothioprine, Methotrexate, and Copaxone; agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g., agonists, antagonists, such as Rosiglitazone, Troglitazone and Pioglitazone), insulin secretagogues (for example, sulfonylurea drugs, such as Glyburide, Glimepiride, Chlorpropamide, Tolbutamide, and Glipizide, and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors and 11beta-HSD inhibitors), hepatic glucose output lowering compounds (such as glucagon antagonists and metaformin, such as Glucophage and Glucophage XR), insulin and insulin derivatives (both long and short acting forms and formulations of insulin), and anti-obesity drugs (such as β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat)).

### EXAMPLES

The following preparations and examples are given to enable those skilled in the art to more clearly understand and to practice the present invention. They should not be considered as limiting the scope of the invention, but merely as being illustrative and representative thereof.

All spectra were recorded at 300 MHz on a Bruker Instruments NMR unless otherwise stated. Coupling constants (J) are in Hertz (Hz) and peaks are listed relative to TMS (δ 0.00 ppm). Microwave reactions were performed using a Personal Chemistry Optimizer™ microwave reactor in 10 mL Personal Chemistry microwave reactor vials. All reactions were performed at 200° C. for 600 s with the fixed hold time ON unless otherwise stated. Sulfonic acid ion exchange resins (SCX) were purchased from Varian Technologies. Analytical HPLC was performed on 4.6 mm×100 mm Waters Sunfire RP C18 5 μm column using (i) a gradient of 20/80 to 80/20 acetonitrile (0.1% formic acid)/water (0.1% formic acid) over 6 min (Method A), (ii) a gradient of 20/80 to 80/20 acetonitrile (0.1% formic acid)/water (0.1% formic acid) over 8 min (Method B), (iii) a gradient of 40/60 to 80/20 acetonitrile (0.1% formic acid)/water (0.1% formic acid) over 6 min (Method C), (iv) a gradient of 40/60 to 80/20 acetonitrile (0.1% formic acid)/water (0.1% formic acid) over 8 min (Method D), or (v) a gradient of 10/60 to 60/10 acetonitrile (0.1% formic acid)/water (0.1% formic acid) over 8 min (Method E). Preparative HPLC was performed on 30 mm×100 mm Xtera Prep RP₁₈ 5μ columns using an 8 min gradient of 95/5 to 20/80 water (0.1% formic acid)/acetonitrile (0.1% formic acid) unless otherwise stated.

### Reference A

### Synthesis of 2-(4-methoxyphenyl)-3-methylmorpholine

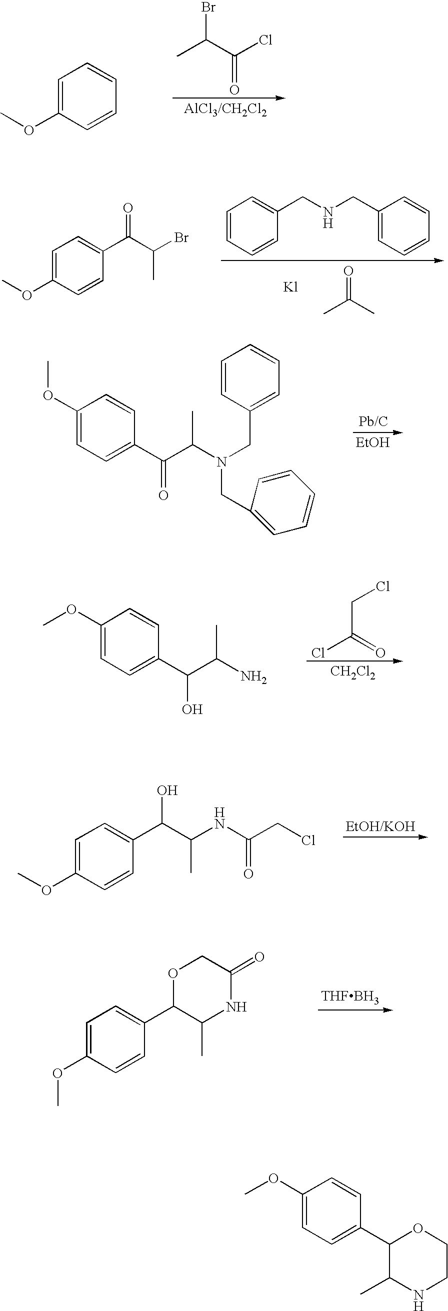

Step 1. Into a 1000 mL 4-necked round bottom flask purged and maintained with an inert atmosphere of nitrogen containing a solution of AlCl₃ (160.2 g, 1.20 mol) in CH₂Cl₂ (50 mL) was added a solution of anisole (64.8 g, 599.44 mmol) in CH₂Cl₂ (50 mL) dropwise with stirring at 0° C. over a 30 minute period. This was followed by the drop-wise addition of a solution of 2-bromopropanoyl chloride (128.5 g, 749.71 mmol) in CH₂Cl₂ (200 mL) with stirring at 0° C. over 60 minutes. The resulting solution was stirred for 0.5 hours at 0° C. and then for 2 hours at room temperature. The reaction mixture was quenched by the addition of 1000 mL of HCl/H₂O/ice and then extracted three times with CH₂Cl₂, the organic fractions were combined, dried over MgSO₄ and concentrated. The residue was purified by silica gel chromatography using 1:100 EtOAc/PE as eluant to provide 25 g of crude 2-bromo-1-(4-methoxyphenyl)propan-1-one as yellow oil.

Step 2. 2-Bromo-1-(4-methoxyphenyl)propan-1-one (12 g, 49.36 mmol), dibenzylamine (19.4 g, 98.33 mmol), acetone (600 mL) and KI (370 mg, 2.23 mmol) were combined in a 1000 mL round bottom flask and stirried for 3 days at room temperature. The reaction mixture was filtered, the filtrate was concentrated and the residue was purified by silica gel chromatography using 1:100 EtOAc/PE as eluant to provide 12.8 g (58%) of 2-(dibenzylamino)-1-(4-methoxyphenyl)propan-1-one as a white solid.

Step 3. Into a 100 mL round bottom flask purged, flushed and maintained with a hydrogen atmosphere was added 2-(dibenzylamino)-1-(4-methoxyphenyl)propan-1-one (3 g, 8.34 mmol), Pd/C (3 g), EtOH (75 mL) and HCl (0.6 mL). The reaction mixture was stirred overnight at room temperature, filtered and the filtrate was concentrated to provide 1.4 g of 2-amino-1-(4-methoxyphenyl)propan-1-ol as a white solid.

Step 4. Into a mixture of 2-amino-1-(4-methoxyphenyl)propan-1-ol (3.1 g, 17.11 mmol), NaOH (1.0 g), 5 drops of water and CH₂Cl₂ (mL) was added a solution of 2-chloroacetyl chloride (2.9 g, 25.7mmol) in CH₂Cl₂ (15 mL) drop-wise with stirring at 0° C. over a 15 minute period. The reaction mixture was stirred for 1.5 hours at 0° C. in a bath of H₂O/ice and then washed with HCl/H₂O, NaHCO₃/H₂O, dried over MgSO₄ and concentrated to provide 3.3 g of 2-chloro-N-(1-hydroxy-1-(4-methoxyphenyl)propan-2-yl)acetamide as a white solid.

Step 5. 2-Chloro-N-(1-hydroxy-1-(4-methoxyphenyl)propan-2-yl)acetamide (670 mg, 2.60 mmol), KOH (0.56 g) and EtOH (70 mL) were combined and stirred for 2.5 hours at room temperature. The reaction mixture was concentrated, diluted with 10 mL of H₂O and extracted with CH₂Cl₂. The organic layers were combined, dried over MgSO₄ and concentrated to provide 0.33 g of 6-(4-methoxyphenyl)-5-methylmorpholin-3-one as a white solid.

Step 6. A solution of 6-(4-methoxyphenyl)-5-methylmorpholin-3-one (330 mg, 1.49 mmol) in THF (50 mL) contained in a 100 mL 3-necked round bottom flask purged and maintained with an inert atmosphere of nitrogen was treated with THF.BH₃ (15 mL) in several batches while cooling to 0° C. over a period of 10 minutes. Stirring was continued for 3 hours at room temperature and the reaction progress was monitored by TLC (CH₂Cl₂/MeOH=10:1). The reaction mixture was quenched by adding 10 mL of MeOH. The reaction mixture was concentrated, diluted with 30 mL of 10% HCl/H₂O and warmed to 80° C. for 0.5 hours. The pH was adjusted to 10 by the addition of NaOH (20% aq. solution), extracted with EtOAc, dried over Na₂SO₄ and concentrated to provide 0.3 g of 2-(4-methoxyphenyl)-3-methylmorpholine as a light yellow liquid. LCMS [M+H]⁺ calcd for C₁₂H₁₈NO₂ 208, found 208.

### Reference B

### Synthesis of 4-bromo-6,7-dimethoxycinnoline

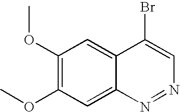

Step 1. 1-(2-Amino-4,5-dimethoxyphenyl)ethanone (15.60 g, 79.91 mmol) was dissolved in concentrated hydrogen chloride in water (555 mL) and water (78 mL). The mixture was cooled to −5° C. and a solution of sodium nitrite (5.55 g, 80.4 mmol) in water (20 mL) was added over a period of 45 minutes. The mixture was stirred for an additional 1 h at 0° C. and then warmed to 60-75° C. for 4 h. The mixture was then cooled to room temperature using an ice bath and the resulting precipitate was collected via filtration. The solid hydrochloride salt thus obtained was added to approximately 1.0 L of water and then basified to pH ˜12 with sodium hydroxide. The brown solution was neutralized with hydrochloric acid, and the resulting precipitate was collected to provide 12.77 g of 6,7-dimethoxycinnolin-4-ol as a light tan solid (78% yield), which was used without further purification. MS [M+H]=207. ¹H NMR (DMSO d₆) δ (ppm) 7.62 (s, 1H), 7.30 (s, 1H), 6.93(s, 1H), 3.89 (s, 3H), 3.85 (s, 3H).

Step 2. To a solution of 6,7-dimethoxycinnolin-4-ol (2.00 g, 9.70 mmol, prepared as described above in step 1) in chloroform (20 mL) was added phosphorus oxybromide (12.2 g, 0.0426 mol). Brief solvation was observed for 10 minutes after addition of the phosphorus oxybromide then a suspension formed. The mixture was stirred for 8 h at room temperature, and was then heated to reflux for 18 h. The mixture was poured onto crushed ice (resulting in gas evolution), warmed to room temperature (giving a volume of around 125 mL) and neutralized to ˜pH 7 with saturated sodium acetate. The mixture was then extracted with dichloromethane (5×50 mL) and the combined organics were dried (MgSO₄), filtered, and concentrated. Re-crystallization from absolute ethanol provided 1.30 g of 4-bromo-6,7-dimethoxycinnoline (50% yield) as light yellow superfine fibrous crystals. MS [M+]=269, [M+2]=271, ¹H NMR (DMSO d6) δ (ppm) 9.38 (s, 1H) 7.77 (s, 1H), 7.21 (s, 1H), 4.03 (s, 6H).

### Reference C

### Synthesis of 1-(6,7-dimethoxycinnolin-4-yl)piperidin-4-amine

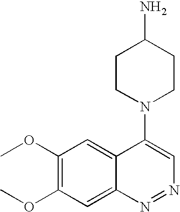

A mixture of 4-bromo-6,7-dimethoxycinnoline (0.5 g, 0.002 mol), 4-BOC-amino-piperidine (0.5619 g, 2.806 mmol), tris(dibenzylideneacetone)dipalladium(0) (0.0891 g, 0.0973 mmol), 9,9-dimethyl-4,5-bis(diphenylphosphino)xanthene (0.110 g, 0.190 mmol), sodium tert-butoxide (0.268 g, 2.79 mmol) and toluene (4.0 mL, 0.037 mol) was heated at 50° C. overnight. The reaction mixture was flushed through an SCX column, washed with methanol and eluted with 2.0 M ammonia/methanol. The product was purified by silica gel chromatography on a 40 g column using a gradient going from 100% CH₂Cl₂ to 50% (8:1:1 CH₂Cl₂/MeOH/7M NH₃ in MeOH)/CH₂Cl₂ as elutant to provide 1-(6,7-dimethoxycinnolin-4-yl)piperidin-4-amine.

### Example 1

### Synthesis of 4-(1,3-benzoxazol-2-yl)-6,7-dimethoxycinnoline hydroformate

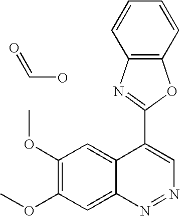

n-Butyllithium (0.0639 g, 0.997 mmol) was added dropwise over 30 minutes to a chilled (−30° C.) solution of benzoxazole (0.119 g, 0.997 mmol) in N,N-dimethylacetamide (3 mL). Tris(dibenzylideneacetone)dipalladium(0) (0.046 g, 0.050 mmol) and a solution of 4-bromo-6,7-dimethoxycinnoline (0.134 g, 0.498 mmol, prepared as described in Reference B above) in N,N-dimethylacetamide (3 mL) was added. The resulting mixture was heated to 85° C. for 8 h, then cooled to room temperature. The solvent was evaporated and the residue was diluted with ethyl acetate (30 mL). The solution was filtered through celite, washed with aqueous sodium bicarbonate (20 mL), and then concentrated. The crude product was purified by column chromatography (gradient elution using 0-5% methanol/dichloromethane) followed by preparative HPLC to give 0.02 g of 4-(1,3-benzoxazol-2-yl)-6,7-dimethoxycinnoline hydroformate (13% yield). ¹H NMR (CDCl₃) δ (ppm) 9.88 (s, 1H), 8.91 (s, 1H), 7.93 (d, J=7.2 Hz, 1H), 7.87 (s, 1H), 7.72 (d, J=7.5 Hz, 1H), 7.49 (m, 2H), 4.24 (s, 3H), 4.16 (s, 3H), LC/MS (EI) t_{R} 7.10 min (Method B), m/z 308.1 (M⁺+1).

### Example 2

### Synthesis of N-cyclopropyl-1-(6,7-dimethoxycinnolin-4-yl)-1H-indazole-3-carboxamide hydroformate

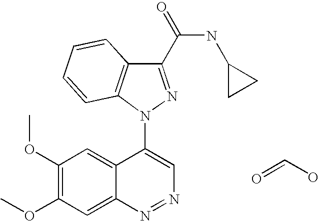

Step 1. n-Butyllithium (0.13 g, 0.0020 mol) was added dropwise over 30 minutes to a chilled (−30° C.) solution of 1H-indazole-3-carboxylic acid (0.162 g, 0.999 mmol) in N,N-dimethylacetamide (3 mL). A solution of tris(dibenzylideneacetone)dipalladium(0) (0.083 g, 0.091 mmol), 4-bromo-6,7-dimethoxycinnoline (0.244 g, 0.908 mmol, prepared as describe above in Example 1) and triethylamine (380 μL) in N,N-dimethylacetamide (3 mL) was added and the reaction mixture was raised to 25° C. for 5 minutes, then to 85° C. for 2 hours. The solvent was removed by evaporation and the residue was diluted with 20% methanol/dichloromethane (50 mL), filtered through celite and concentrated. Purification by column chromatography (gradient elution using 30-60% methanol/ethyl acetate) gave 0.318 g 1 -(6,7-dimethoxycinnolin-4-yl)-1H-indazole-3-carboxylic acid (42.4% yield). A 10 mg portion of the purified product was further purified by preparative HPLC. LC/MS (EI) t_{R} 5.65 min (Method B), m/z 351.1 (M⁺+1).

Step 2. A mixture of 1-(6,7-dimethoxycinnolin-4-yl)-1H-indazole-3-carboxylic acid (30 mg, 0.08 mmol, prepared as in Step 1 above), cyclopropylamine (0.00978 g, 0.171 mol), N,N′-diisopropylcarbodiimide (21.4 μL), 1-hydroxybenzotriazole (5.8 mg, 0.043 mol), and N,N-dimethylformamide (2.00 mL) was stirred at room temperature for 8 h. The solvent was then evaporated. The resulting residue was dissolved in ethyl acetate (50 mL), and the solution was washed with aqueous sodium bicarbonate and concentrated. Purification by preparative HPLC gave 0.009 g of N-cyclopropyl-1-(6,7-dimethoxycinnolin-4-yl)-1H-indazole-3-carboxamide hydroformate as light yellow solid (30% yield). ¹H NMR (CDCl₃) δ (ppm) 9.34 (b, 1H), 8.59 (d, J=7.5 Hz, 1H), 7.91 (s, 1H), 7.55-7.30 (m, 3H), 7.06 (s, 1H), 4.16 (s, 3H), 3.87 (s, 3H), 3.00 (b, 1H), 0.93 (d, J=8.0 Hz, 2H), 0.71 (s, 2H), LC/MS (EI) t_{R} 6.34 min (Method B), m/z 390.1 (M⁺+1).

### Example 3

### Synthesis of 6,7-dimethoxy-4-[4-(2-methoxyethoxy)-1H-indazol-1-yl]cinnoline

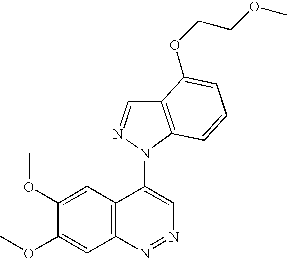

Into a 5 mL microwave tube was added 4-bromo-6,7-dimethoxycinnoline (200 mg, 0.743 mmol, prepared as described in Reference B above), 4-(2-methoxyethoxy)-1H-indazole (171.0 mg, 0.8895 mmol), copper (I) iodide (28 mg, 0.15 mmol), potassium carbonate (206.7 mg, 1.496 mmol), N,N′-dimethyl-1,2-ethanediamine (32 μL) and toluene (6.00 mL). The resulting dark, olive-green colored suspension was heated at 115° C. for 24 h. The crude product was purified by preparative HPLC (using a gradient elution 10:90 to 80:20 acetonitrile:water with 0.1% formic acid and a flow rate of 45 mL/min) to give 0.031 g of 6,7-dimethoxy-4-[4-(2-methoxyethoxy)-1H-indazol-1-yl]cinnoline (11% yield). ¹H NMR (CDCl₃) δ (ppm) 9.38 (s, 1H), 8.29 (s, 1H), 7.87 (s, 1H), 7.74 (d, J=8.4 Hz, 1H), 7.52 (s, 1H), 7.05 (d, J=8.9 Hz, 1H), 6.90 (s, 1H), 4.15 (s, 3H), 3.96 (s, 3H), 3.87 (m, 4H), 3.21 (m, 4H), LC/MS (EI) t_{R} 6.39 min (Method B), m/z 381 (M⁺+1).

The following compounds were prepared in a similar manner to Example 4 using different starting materials:

### 6,7-Dimethoxy-4-[5-(2-methoxyethoxy)-1H-indazol-1-yl]cinnoline:

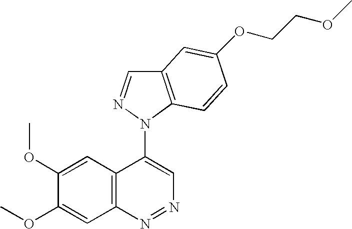

Prepared using 5-(2-methoxyethoxy)-1H-indazole to give 25 mg of above compound. LC/MS (EI) t_{R} 6.12 min (Method B), m/z 381 (M⁺+1).

### 6,7-Dimethoxy-4-[6-(2-methoxyethoxy)-1H-indazol-1-yl]cinnoline:

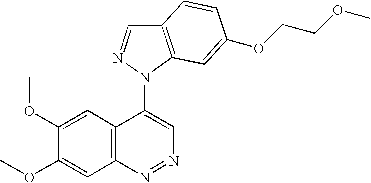

Prepared using 6-(2-methoxyethoxy)-1H-indazole to give 15 mg of above compound. LC/MS (EI) t_{R} 6.23 min (Method B), m/z 381 (M⁺+1).

### 6,7-Dimethoxy-4-(5-pyridin-3-yl-1H-indazol-1-yl)cinnoline:

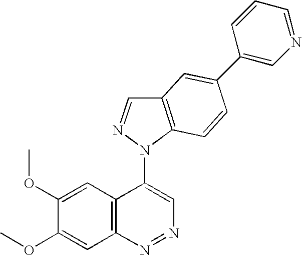

Prepared using 5-pyridin-3-yl-1H-indazole to give 2 mg of above compound. LC/MS (EI) t_{R} 4.39 min (Method B), m/z 384 (M⁺+1).

### 6,7-Dimethoxy-4-(5-pyridin-4-yl-1H-indazol-1-yl)cinnoline:

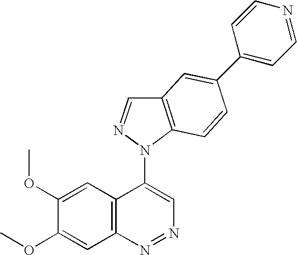

Prepared using 5-pyridin-4-yl-1H-indazole to give 2 mg of above compound. LC/MS (EI) t_{R} 3.89 min (Method B), m/z 384 (M⁺+1).

### Example 4

### Synthesis of 6,7-dimethoxy-4-(6-morpholin-4-yl-1H-indazol-1-yl)cinnoline

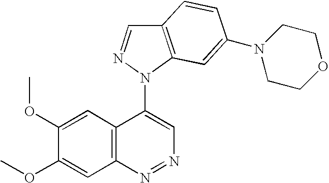

Step 1. Into a 5 mL microwave tube was added 4-bromo-6,7-dimethoxycinnoline (250 mg, 0.743 mmol, prepared as described in Example 1 above), 6-bromo-1H-indazole (219.1 mg, 1.112 mmol), copper(I) iodide (18 mg, 0.093 mmol), potassium carbonate (258.4 mg, 1.870 mmol), N,N′-dimethyl-1,2-ethanediamine (40 μL) and toluene (1 mL) The resulting dark, olive-green colored suspension was heated at 115° C. for 24 h. The crude product was purified by flash chromatography on silica gel (using a gradient of 50% ethyl acetate/hexanes to 100% hexanes) to give 0.342 g of 4-(6-bromo-1H-indazol-1-yl)-6,7-dimethoxycinnoline (95.6% yield) which was used in the next step without further purification.

Step 2. Into a 10 ml sealed microwave tube was added 4-(6-bromo-1H-indazol-1-yl)-6,7-dimethoxycinnoline (100 mg, 0.260 mmol, prepared as described in step 1 above, morpholine (34.0 μL, 0.389 mmol), tetrahydrofuran (5.0 mL), tris(dibenzylideneacetone)dipalladium(0) (24 mg, 0.026 mmol), 9,9-dimethyl-4,5-bis(diphenylphosphino)xanthene (22 mg, 0.039 mmol), sodium tert-butoxide (74.8 mg, 0.779 mmol), and the resulting mixture was heated to 70° C. for 12 h. The crude product was purified by preparative HPLC (using a gradient elution 10:90 to 80:20 acetonitrile:water with 0.1% formic acid and a flow rate of 45 mL/min) to give 6 mg of 6,7-dimethoxy-4-(6-morpholin-4-yl-1H-indazol-1-yl)cinnoline (6% yield). ¹H NMR (CDCl₃) δ (ppm) 9.36 (s, 1H), 8.53 (s, 1H), 7.85 (s, 1H), 7.44 (s, 1H), 7.40 (m, 1H), 7.16 (d, J=8.1 Hz 1H), 6.66 (d, J=7.6 Hz, 1H), 4.36 (m, 2H), 4.14 (s, 3H), 3.93 (s, 3H), 3.90 (m, 2H), 3.53 (s, 3H), LC/MS (EI) t_{R} 6.03 min (Method B), m/z 692 (M⁺+1).

The following compound was prepared in a similar manner to Example 4 using different starting materials:

### 6,7-Dimethoxy-4-(5-morpholin-4-yl-1H-indazol-1-yl)cinnoline:

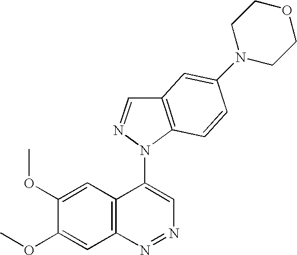

Prepared using 5-bromo-1H-indazole to give above compound. LC/MS (EI) t_{R} 5.72 min (Method B), m/z 392 (M⁺+1).

### Example 5

### Synthesis of 6,7-dimethoxy-4-(4-morpholin-4-yl-1H-indazol-1-yl)cinnoline

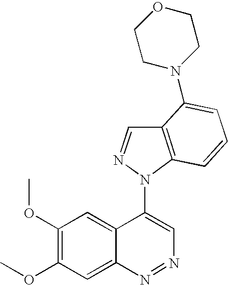

Step 1. n-Butyllithium (0.0704 g, 1.10 mmol) was added dropwise over 30 minutes to a chilled (−30° C.) solution of 4-bromo-1H-indazole (0.197 g, 1.00 mmol) in N,N-dimethylacetamide (3 mL). To this was added a mixture of tris(dibenzylideneacetone)dipalladium(0) (0.04 g, 0.05 mmol), 4-bromo-6,7-dimethoxycinnoline (0.269 g, 1.00 mmol) and triethylamine (420 μL) in N,N-dimethylacetamide (3 mL). The temperature of the reaction was raised to ° C. for 5 minutes, then to 85° C. for 12 h. The solvent was then evaporated and the residue was diluted with 10% methanol/dichloromethane (100 mL) and filtered through celite. The solution was concentrated and purified by column chromatography (using a gradient of 3-6% methanol/dichloromethane as eluent), followed by preparative HPLC to afford 120 mg (31.2% yield) of 4-(4-bromo-1H-indazol-1-yl)-6,7-dimethoxycinnoline as an off-white solid. m/z 385.0 (M⁺+1).

Step 2. A mixture of 4-(4-bromo-1H-indazol-1-yl)-6,7-dimethoxycinnoline (25 mg, 0.065 mmol, prepared as described in Step 1 above), morpholine (10.2 mL, 0.117 mmol), tris(dibenzylideneacetone)dipalladium(0) (7.1 mg, 0.0078 mmol), 9,9-dimethyl-4,5-bis(diphenylphosphino)xanthane (6.8 mg, 0.012 mmol), tetrahydrofuran (2.0 mL) and sodium tert-butoxide (22.4 mg, 0.234 mol) was heated at 82° C. for 12 hours. After cooling to room temperature, the mixture was diluted with 10% methanol/dichloromethane (50 mL) and filtered through celite. The filtrate was concentrated and the product was purified by column chromatography (using a gradient of 3-5% methanol/dichloromethane as eluent) followed by preparative TLC to afford 15 mg (59% yield) of 6,7-dimethoxy-4-(4-morpholin-4-yl-1H-indazol-1-yl)cinnoline as a light yellow solid. ¹H NMR (CDCl₃) δ (ppm) 9.35 (s, 1H), 8.41 (s, 1H), 7.86 (s, 1H), 7.43-7.37 (m, 2H), 7.16 (d, J=8.4 Hz, 1H), 6.70 (d, J=7.5 Hz, 1H), 4.15 (s, 3H), 4.01 (t, J=4.5 Hz, 4H), 3.94 (s, 3H), 3.39 (t, J=4.5 Hz, 4H), LC/MS (EI) t_{R} 6.6 min (Method B), m/z 392.1 (M⁺+1).

The following compound was prepared in a similar manner to Example 5 using different starting materials:

### 6,7-Dimethoxy-4-[4-(4-methylpiperazin-1-yl)-1H-indazol-1-yl]cinnoline:

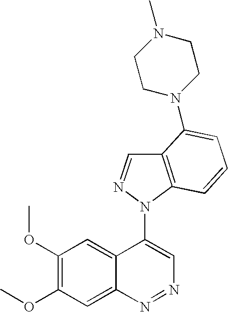

Prepared using 1-methyl-piperazine in Example 5, Step 2 above, to give 8.0 mg of above compound. LC/MS (EI) t_{R} 4.76 min (Method E), m/z 476 (M⁺+1).

### Example 6

### Synthesis of 4-(2,3-dihydro-1,4-benzodioxin-6-yl)-6,7-dimethoxycinnoline

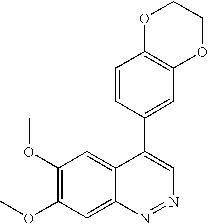

Into a 5 mL microwave tube was added 4-bromo-6,7-dimethoxycinnoline (50.3 mg, 0.187 mmol), 1,4-benzodioxane-6-boronic acid (38.6 mg, 0.214 mmol), bis(triphenylphosphine)-palladium(II) chloride (26.2 mg, 0.0373 mmol), sodium carbonate (2.00 M solution in water, 140 μL) and a mixture of 1,2-dimethoxyethane:water:ethanol (7:3:2 ratio, 900 μL). The resulting brown suspension was subjected to microwave radiation at a temperature of 140° C. for 5.0 minutes. The mixture was then filtered through celite, which was washed with ethyl acetate (20 mL). The organics were combined and washed with water (20 mL, to which a few drops of brine were added), and then washed with brine (15 mL). The organic layer was loaded onto an SCX column (0.5 g). The SCX column was rinsed several times with two column volumes of methanol and the product was eluted using 7.0 M ammonia in methanol (5 mL). Volatiles were removed in vacuo to afford 59.7 mg of a yellow solid which contained 4.8 wt % dichloromethane. Tetrahydrofuran (0.5 mL) and ether (0.1 mL) were then added and removed in vacuo to give 56.8 mg of 4-(2,3-dihydro-1,4-benzodioxin-6-yl)-6,7-dimethoxycinnoline as a yellow solid (which contained 1.7 wt % tetrahydrofuran by ¹H NMR). ¹H NMR (CDCl₃) d 9.03 (s, 1 H), 7.79 (s, 1 H), 6.93 (s, 1 H), 7.22 (s, 1 H), 7.10 (s, 1 H), 7.06 (s, 2 H), 4.37 (s, 4 H), 4.12 (s, 3 H), 3.96 (s, 3 H). LC/MS (EI) t_{R} 4.0 min (Method D), m/z 325.1 (M⁺+1).

The following compounds were prepared in a similar manner to Example 6 using different starting materials:

### 4-(1,3-benzodioxol-5-yl)-6,7-dimethoxycinnoline:

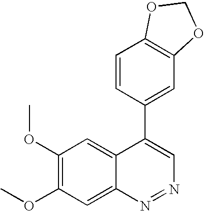

Prepared using 3,4-methylenedioxyphenylboronic acid. Purification did not involve the addition and removal of tetrahydrofuran/ether (8.4 mg, 10% yield). LC/MS (EI) t_{R}4.0 min (Method D), m/z 311.1 (M⁺+1).

### 7-(6,7-dimethoxycinnolin-4-yl)-4-methyl-3,4-dihydro-2H-1,4-benzoxazine:

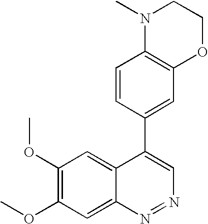

Prepared using 4-methyl-7-(4,4,5,5-tetramethyl-1,3,2-dioxaboralan-2-yl)-3,4-dihydro-2H-1,4-benzoxazine. Purification by column chromatography using a gradient of chloroform to 10% methanol in chloroform afforded 62 mg (98% yield). LC/MS (EI) t_{R} 3.17 min (Method D), m/z 338 (M⁺+1).

### 6,7-Dimethoxy-4-(4-pyridin-4-yl-1H-indazol-1-yl)cinnoline:

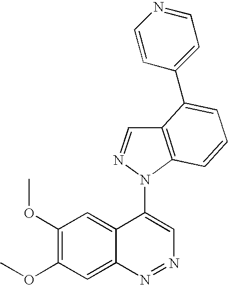

Prepared using pyridine-4-boronic and 4-(4-bromo-1H-indazol-1-yl)-6,7-dimethoxycinnoline (prepared as described above in Example 5, Step 1). Purification by preparative HPLC afforded 13 mg (52% yield). LC/MS (EI) t_{R} 4.78 min (Method B), m/z 384.1 (M⁺+1).

### Example 7

### Synthesis of 6,7-dimethoxy-4-(2-phenylmorpholin-4-yl)cinnoline

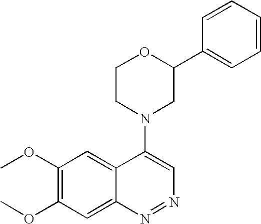

Into a 10 ml sealed microwave tube was added 4-bromo-6,7-dimethoxycinnoline (99.9 mg, 0.371 mmol), 2-phenylmorpholine hydrochloride (89.6 mg, 0.449 mmol), tris(dibenzylideneacetone)dipalladium(0) (20.4 mg, 0.0223 mmol), 9,9-dimethyl-4,5-bis(diphenylphosphino)xanthane (22.8 mg, 0.0394 mmol), sodium tert-butoxide (93.8 mg, 0.976 mmol) and toluene (3 mL). The resulting red-brown suspension was stirred at 50° C. overnight, and then filtered through celite, which was washed with ethyl acetate (20 mL). The combined organics were concentrated, and the crude product was purified by column chromotography (using a gradient elution of 20-80% acetonitrile:water with 0.1% formic acid and a flow rate of 45 mL/min). The organic layer was loaded onto an SCX column (1.0 g). The SCX column was rinsed once with two column volumes of methanol and the product was eluted using 7.0 M ammonia in methanol (8 mL). Volatiles were removed in vacuo to afford 48.3 mg of 6,7-dimethoxy-4-(2-phenylmorpholin-4-yl)cinnoline (37.0% yield). ¹H NMR (CDCl₃) d 8.81 (s, 1 H), 7.71 (s, 1 H), 7.40 (m, 5 H), 7.17 (s, 1 H), 4.88 (d, J=9.0 Hz, 1 H), 4.28 (d, J=12.0 Hz, 1 H), 4.17 (d, J=12.0 Hz, 1 H), 4.09 (s, 6 H), 3.64 (d, J=12.0 Hz, 1 H), 3.52 (d, J=12.0 Hz, 1 H), 3.29 (t, J=10.5 Hz, 1 H), 3.07 (t, J=12.07 Hz, 1 H). LC/MS (EI) t_{R} 4.2 min (Method B), m/z 352.1 (M⁺+1).

The following compounds were prepared in a similar manner to Example 7 using different starting materials:

### 6,7-Dimethoxy-4-[2-(4-methoxyphenyl)morpholin-4-yl]cinnoline:

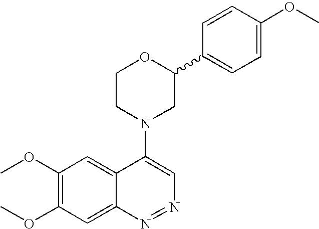

Prepared using 2-(4-methoxyphenyl)morpholine to give 23.9 mg of above compound. LC/MS (EI) t_{R} 4.2 min (Method B), m/z 382.2 (M⁺⁺¹).

### 4-[2-(4-Fluorophenyl)morpholin-4-yl]-6,7-dimethoxycinnoline:

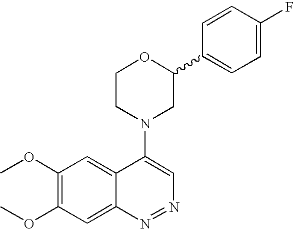

Prepared using 2-(4-fluorophenyl)morpholine to give 5.3 mg of above compound. LC/MS (EI) t_{R} 4.5 min (Method B), m/z 370.1 (M⁺+1).

### 4-[2-(2-Chlorophenyl)morpholin-4-yl]-6,7-dimethoxycinnoline:

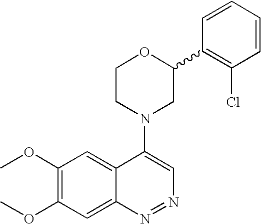

Prepared using 2-(2-chlorophenyl)morpholine oxalate to give 9.5 mg of above compound. LC/MS (EI) t_{R} 4.7 min (Method B), m/z 386.1 (M⁺+1).

### 6,7-Dimethoxy-4-(4-phenoxypiperidin-1-yl)cinnoline:

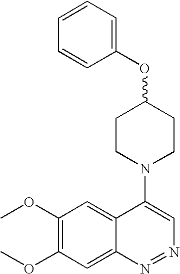

Prepared using 4-phenoxypiperidine hydrochloride to give 42.1 mg of above compound. LC/MS (EI) t_{R} 4.4 min (Method B), m/z 366.2 (M⁺+1).

### 6,7-Dimethoxy-4-(2-pyridin-3-ylmorpholin-4-yl)cinnoline:

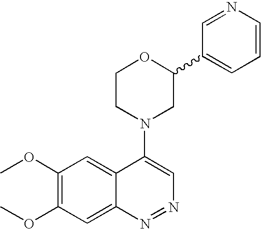

Prepared using 2-pyridin-3-yl-morpholine oxalate to give above compound. The product was further purified by separating a dichloromethane solution of the crude product on a Berger Mini-Gram (4.6 mm×250 mm pyridine column with an isocratic 6.0 min run of 10% methanol with 0.1% 1,2-dimethoxyethane and a flow rate of 9.9 mL/min. 8.7 mg (8.1% yield). LC/MS (EI) t_{R} 2.7 min (Method B), m/z 353.1 (M⁺+1).

### 6,7-Dimethoxy-4-(4-methyl-3-phenylpiperazin-1-yl)cinnoline:

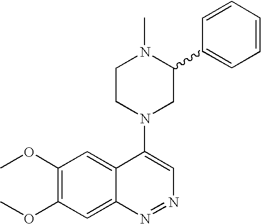

Prepared using 1-methyl-2-phenylpiperazine dihydrochloride. m/z 365.1 (M⁺+1).

### 6,7-Dimethoxy-4-[2-(pyrrolidin-1-ylmethyl)morpholin-4-yl]cinnoline:

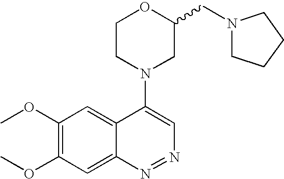

Prepared using 2-(pyrrolidin-1-ylmethyl)morpholine. m/z 359.2 (M⁺+1).

### 1′-(6,7-Dimethoxycinnolin-4-yl)-1,3′-bipiperidin-2-one:

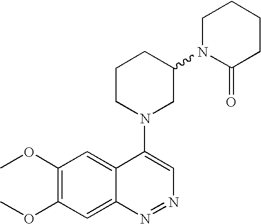

Prepared using 3-(N-delta-valerolactam)piperidine hydrochloride (reaction time of 2.7 days at 50° C.). m/z 371.2 (M⁺+1).

### 6,7-Dimethoxy-4-[3-(pyrrolidin-1-ylcarbonyl)piperidin-1-yl]cinnoline:

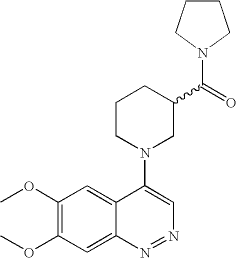

Prepared using 3-(pyrrolidin-1-ylcarbonyl)piperidine (reaction time of 2.7 days at 50° C.). m/z 371.2 (M⁺+1).

### 4-(3-Benzylpiperidin-1-yl)-6,7-dimethoxycinnoline:

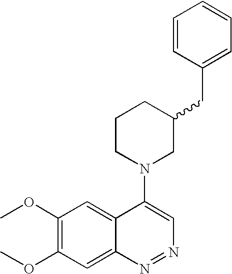

Prepared using 3-benzylpiperidine (reaction time of 2.7 days at 50° C.). m/z 364.2 (M⁺+1).

### Example 8

### Synthesis of 6,7-dimethoxy-4-(3-phenylpyrrolidin-1-yl)cinnoline

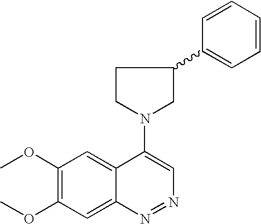

Into a 5 mL microwave tube was added 4-bromo-6,7-dimethoxycinnoline (117.9 mg, 0.4381 mmol) and 3-phenylpyrrolidine (52.9 mg, 0.359 mmol), tris(dibenzylideneacetone)-dipalladium(0) (17.4 mg, 0.0190 mmol), 9,9-dimethyl-4,5-bis(diphenylphosphino)xanthene (31.5 mg, 0.0544 mmol), sodium tert-butoxide (74.3 mg, 0.773 mmol) and toluene (0.7 mL). The resulting yellow-green suspension was stirred at 60° C. overnight. Aqueous hydrogen chloride (0.1 M, 5 mL) was then added, resulting a yellow solution and brown flocculent precipitate. The solution was filtered through celite, and the solution was adjusted to a pH of approximately 11-12, resulting a cloud yellow emulsion. The product was extracted with ethyl acetate (1×20 mL) and the organics were washed with an aqueous saturated solution of sodium bicarbonate (1×15 mL). The organic layer was dried over sodium sulfate, filtered, and concentrated in vacuo to afford an orange oil. The flocculent brown precipitate was extracted with warm acetonitrile (20 mL) and the solution was filtered through celite and then concentrated. The combined products were purified by preparative HPLC (using a gradient elution 20-80% acetonitrile:water with 0.1% formic acid and a flow rate of 45 mL/min). The organic layer was then loaded onto an SCX column (0.5 g). The SCX column was rinsed approximately 2 mL of methanol and the product was eluted using 2 M ammonia in methanol (70 mL). Volatiles were removed in vacuo to afford 23.6 mg of 6,7-dimethoxy-4-(3-phenylpyrrolidin-1-yl)cinnoline as a yellow-green solid (19.6% yield). ¹H NMR (CDCl₃) d 8.53 (s, 1 H), 7.65 (s, 1 H), 7.50 (m, 5 H), 4.13 (m, 1 H), 4.07 (s, 3 H), 3.97 (s, 3 H), 3.89 (m, 2 H), 3.60 (qn, J=6.0 Hz, 1 H), 2.51 (m, 1 H), 2.26 (qn, J=9.0 Hz, 1 H), 2.01 (s, 1 H). LC/MS (EI) t_{R} 4.2 min (Method B), m/z 336.2 (M⁺+1).

### Example 9

### Synthesis of 4-[5-(benzyloxy)-1H-indazol-1-yl]-6,7-dimethoxycinnoline

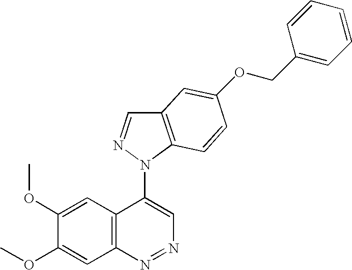

Into a 10 ml sealed microwave tube was added 4-bromo-6,7-dimethoxycinnoline (250 mg, 0.929 mmol), 5-(benzyloxy)-1H-indazole (189 mg, 0.844 mmol), toluene (5.0 mL), tris(dibenzylideneacetone)dipalladium(0) (40 mg, 0.04 mmol) 9,9-dimethyl-4,5-bis(diphenylphosphino)xanthene (49 mg, 0.084 mmol), and sodium tert-butoxide (240 mg, 2.5 mmol) and the reaction was heated to at 80° C. for 12 h. The crude product was purified by preparative HPLC (using a gradient elution 10:90 to 80:20 acetonitrile:water with 0.1% formic acid and a flow rate of 45 mL/min). The product was further purified on a Berger SFC Minigram instrument using 10% methanol (with 0.4% dimethylethylamine) modifier on a pyridine column (7.8×250 mm) at a pressure of 120 bar, a flow rate of 9.9 mL/min and a column temperature of 35° C., to afford 11 mg of 4-[5-(benzyloxy)-1H-indazol-1-yl]-6,7-dimethoxycinnoline (3.2% yield). m/z 413 (M⁺+1).

The following compounds were prepared in a similar manner to Example 9 using different starting materials:

### 4-[6-(Benzyloxy)-1H-indazol-1-yl]-6,7-dimethoxycinnoline:

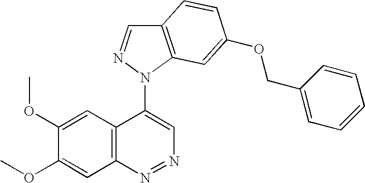

Prepared using 6-(benzyloxy)-1H-indazole to give 23 mg of above compound. m/z 413 (M⁺+1).

### 4-(1H-Indazol-1-yl)-6,7-dimethoxycinnoline:

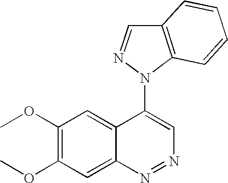

Prepared using 1H-indazole to give 38 mg of above compound. m/z 307 (M⁺+1).

### Example 10

### Synthesis of 6,7-dimethoxy-4-[(2S)-2-phenylmorpholin-4-yl]cinnoline and 6,7-dimethoxy-4-[(2R)-2-phenylmorpholin-4-yl]cinnoline

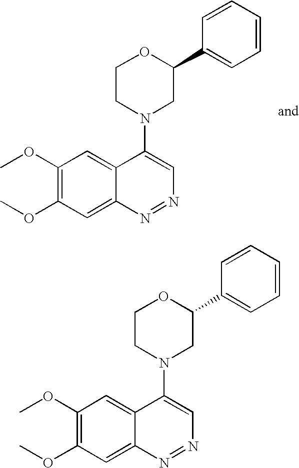

Racemic 6,7-dimethoxy-4-(2-phenylmorpholin-4-yl)cinnoline was prepared as described in Example 7 above. Resolution of this compound into the two enantiomeric forms was accomplished as follows.

6,7-dimethoxy-4-(2-phenylmorpholin-4-yl)cinnoline (40.6 mg) was dissolved in methanol (1.0 mL) and dichloromethane (0.4 mL). The resulting solution was resolved on a Berger SFC Mini-Gram instrument using a 4.6 mm×250 mm Chiral OJ column with an isocratic 6.0 min run of 39% methanol (no dimethoxyethane) in liquid carbon dioxide with a flow rate of 9.9 mL/min. Product collection was by forced time windows at a wavelength of 240 nm. The fraction collected between 3.3 and 4.6 min contained 18.0 mg of 6,7-dimethoxy-4-[(2S)-2-phenylmorpholin-4-yl]cinnoline (99+% enantiomeric excess), LC/MS (EI) t_{R} 4.2 min (Method B), m/z 352.1 (M⁺+1). The fraction collected between 4.6 and 5.6 min contained 17.9 mg of 6,7-dimethoxy-4-[(2R)-2-phenylmorpholin-4-yl]cinnoline (99+% enantiomeric excess), LC/MS (EI) t_{R} 4.2 min (Method B), m/z 352.1 (M⁺+1).

The following compounds were prepared in a similar manner to Example 10 using different starting materials:

### (3′R)-1′-(6,7-dimethoxycinnolin-4-yl)-1,3′-bipiperidin-2-one and (3′S)-1′-(6,7-dimethoxycinnolin-4-yl)-1,3′-bipiperidin-2-one:

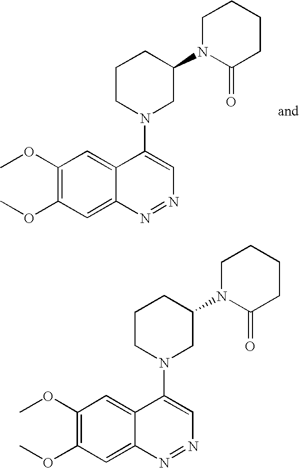

Racemic 1′-(6,7-dimethoxycinnolin-4-yl)-1,3′-bipiperidin-2-one was prepared as described above in Example 7. Using a similar resolution procedure (5.9 min run of 40% methanol with 0.5% dimethoxyethane), the fraction collected between 3.7 and 4.4 min contained 12.7 mg of (3′R)-1′-(6,7-dimethoxycinnolin-4-yl)-1,3′-bipiperidin-2-one (99+% enantiomeric excess), LC/MS (EI) t_{R} 3.35 min (Method B), m/z 371.2 (M⁺+1). The fraction collected between 4.5 and 5.4 min contained 14.4 mg of (3′S)-1′-(6,7-dimethoxycinnolin-4-yl)-1,3′-bipiperidin-2-one (94-98% enantiomeric excess), LC/MS (EI) t_{R} 3.35 min (Method B), m/z 371.2 (M⁺+1).

### Example 11

### Synthesis of 6,7-dimethoxy-4-(5-pyridin-4-yl-1H-indazol-1-yl)cinnoline

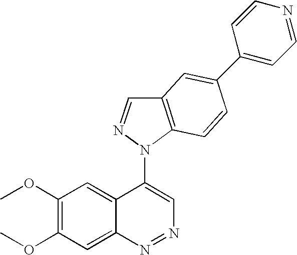

Into a 5 mL microwave tube was added 4-bromo-6,7-dimethoxycinnoline (230 mg, 0.856 mmol), 5-pyridin-4-yl-1H-indazole (200 mg, 1.02 mmol), copper(I) iodide (33 mg, 0.17 mmol), potassium carbonate (238.1 mg, 1.723 mmol), N,N′-dimethyl-1,2-ethanediamine (36 μL, 0.34 mmol) and toluene (6.91 mL). The dark, olive-green colored suspension was heated at 115° C. for 24 hours. The material was diluted in 100 mL of 5% MeOH in DCM and filtered through a pad a celite and washed with DCM. The combined filtrate was collected and washed with 2×30 mL of brine and dried over Na₂SO₄. The crude product was purified by C18 reverse phase preparative HPLC using CH₃CN:H₂O with 0.1% formic acid as solvent system in a gradient elution going from 10:90 to 80:20 at a flow rate of 45 mL/minute to provide 6,7-dimethoxy-4-(5-pyridin-4-yl-1H-indazol-1-yl)cinnoline.

### Example 12

### Synthesis of 4-(3-benzylpyrrolidin-1-yl)-6,7-dimethoxycinnoline

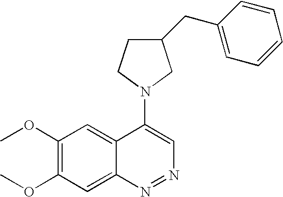

Into a 10 mL sealed microwave tube was added 4-bromo-6,7-dimethoxycinnoline (50.0 mg, 0.186 mmol), 3-benzylpyrrolidine (36.0 mg, 0.223 mmol), toluene (1.5 mL, 0.014 mol), tris(dibenzylideneacetone)dipalladium(0) (8.0 mg, 0.0087 mmol), 9,9-dimethyl-4,5-bis(diphenylphosphino)xanthene (11 mg, 0.019 mol) and sodium tert-butoxide (26.8 mg, 0.279 mol). The reaction mixture was heated to 50° C. for 18 h and then was loaded onto a 10 g SCX column and pushed through with MeOH (1 volume). Elution with NH₃ in MeOH, followed by concentration on the rotovap provided the crude product. Purification by rotary chromatography using a gradient elution going from 100% chloroform to 10% methanol in chloroform provided 51 mg of 4-(3-benzylpyrrolidin-1-yl)-6,7-dimethoxycinnoline.

### Example 13

### Synthesis of 4-[2-(4-fluorophenyl)-2-methylmorpholin-4-yl]-6,7-dimethoxycinnoline

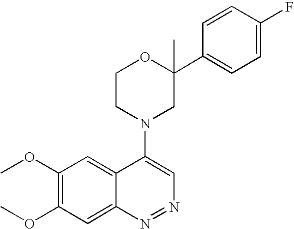

Into a flame-dried 5 mL microwave tube under argon was added 4-bromo-6,7-dimethoxycinnoline (84.1 mg, 0.312 mmol), commercially available 2-(4-fluorophenyl)-2-methyl-morpholine (49.9 mg, 0.256 mol), tris(dibenzylideneacetone)-dipalladium(0) (12.1 mg, 0.0132 mmol), 9,9-dimethyl-4,5-bis(diphenylphosphino)xanthene (15.0 mg, 0.0259 mmol), sodium tert-butoxide (35.7 mg, 0.371 mmol) and toluene (0.6 mL, 6 mmol). The resulting brown suspension was stirred at 50° C. overnight and turned to red-pink suspension by morning. The reaction was monitored by LC/MS and upon completion was loaded onto a 1.76 g SCX column, rinsed with methanol (30 mL, 0.7 mol) and product was eluted with 2.0 M ammonia in methanol (˜10 mL) and concentrated (rotovap). The product was purified on a C18 preparative HPLC column (30×100 mm) using a gradient of CH₃CN:H₂O (with 0.1% formic acid) going from 20% CH₃CN to 80% CH₃CN over 8 minutes and a flow rate of 45 mL/min. Detection was performed at wavelength 390 nm and the product had a retention time of 3.5 minutes. The product was loaded onto an SCX column and washed with one column volume of MeOH and then eluted with 2.0 M ammonia in MeOH (15 mL). The solvent was removed under reduced pressure to provide 13.2 mg of 4-[2-(4-fluorophenyl)-2-methylmorpholin-4-yl]-6,7-dimethoxycinnoline as a yellow solid. LC/MS: M+H⁺=384.1

### Example 14

### Synthesis of 4-{4-[4-(cyclopropylmethyl)piperazin-1-yl]-1H-indazol-1-yl}-6,7-dimethoxycinnoline

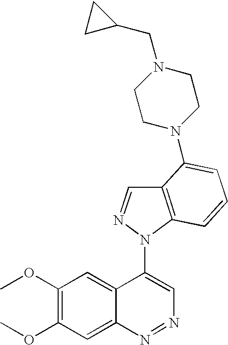

Step 1. A solution of 4-bromo-1H-indazole (0.197 g, 1.00 mmol) in 3 mL of DMA was stirred with n-butyl lithium (0.0704 g, 1.10 mmol) at −30° C. for 30 minutes. A mixture of tris(dibenzylideneacetone)dipalladium(0), 4-bromo-6,7-dimethoxycinnoline (0.269 g, 1.00 mmol) and triethylamine (420 uL, 3.0 mmol) in 3 mL of DMA was added and the temperature of the reaction mixture was raised to 25° C. for 5 minutes and then to 85° C. for 12 hours. The reaction was monitored by LC/MS. Upon completion, the solvent was evaporated and the residue was diluted with 100 mL of 10% MeOH/DCM and filtered through celite. The solution was concentrated and purified by silica gel chromatography using a gradient elution going from 3% to 6% MeOH in DCM. The compound was further purified by preparative HPLC (prep2080, rt 6.73 min) to give 4-(4-bromo-1H-indazol-1-yl)-6,7-dimethoxycinnoline as an off white powder.

Step 2. A mixture of 4-(4-bromo-1H-indazol-1-yl)-6,7-dimethoxycinnoline (0.200 g, 0.000519 mol), piperazine (0.4 g, 0.005 mol), tetrahydrofuran (6.00 mL, 0.0740 mol), 2-dicyclohexyl-phosphino-2′,4′,6′-tri-i-propyl-1,1′-biphenyl (0.035 g, 0.073 mmol), tris(dibenzylideneacetone)-dipalladium(0) (0.035 g, 0.038 mmol) and sodium tert-butoxide (0.150 g, 0.00156 mol) was microwaved at 140° C. for 15 minutes. The resulting mixture was diluted with 30 mL of 5% MeOH/DCM and filtered through a pad of celite. The solution was concentrated and purified by column chromatography using a gradient elution going from 20% to 60% MeOH in DCM to give 4-(4-piperazin-1-yl-1H-indazol-1-yl)-6,7-dimethoxycinnoline as a yellow solid 0.072 g (36%).

Step 3. 6,7-Dimethoxy-4-piperazin-1-yl-1H-indazol-1-yl)cinnoline (20 mg, 0.051 mmol), cyclopropylmethyl bromide (0.010 mL, 0.1 mmol), potassium carbonate (21.2 mg, 0.154 mmol) and DMA (2.0 mL) were combined and the reaction mixture was warmed to 80° C. for 3 hours. The solvent was evaporated and the residue was diluted with DCM (30 mL) and filtered through celite. The filtrate was concentrated and purified by silica gel chromatography using a gradient elution going from 3 to 8% MeOH in EtOAc. Additional purification by preparative HPLC (prep0560, uv 250 nm, rt 4.52 min) provided 4-{4-[4-(cyclopropylmethyl)piperazin-1-yl]-1H-indazol-1-yl}-6,7-dimethoxycinnoline as a yellow solid. LC/MS 635_{—}26 2080_{—}8 min, rt 3.86 min, M+H 445.2, M+2H 223.2.

### Example 15

### Synthesis of 6,7-dimethoxy-4-(4-pyrrolidin-1-yl-1H-indazol-1-yl)cinnoline

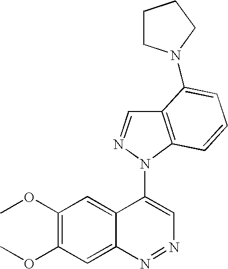

4-(4-Bromo-1H-indazol-1-yl)-6,7-dimethoxycinnoline (45 mg, 0.1.2 mmol), pyrrolidine (33 mg, 0.47 mmol), tetrahydrofuran (4.0 mL), 2-dicyclohexylphosphio-2′,4′,6′-tri-i-propyl-1,1′-biphenyl (8.0 mg, 0.017 mmol), sodium tert-butoxide (44.9 mg, 0.47 mmol) and tris(dibenzylideneacetone)dipalladium(0) (8 mg, 0.09 mmol) were combined in a microwave tube and irradiated in a microwave oven at 300 W to 140° C. for 8.30 minutes. The resulting mixture was diluted with 30 mL of DCM, filtered through celite, concentrated and purified by column chromatography using 1-3% MeOH in 1:1 EtOAc/hexane, DMEA 0.5% as eluant. The product was further purified by HPLC (prep1080, rt 7.23 min, uv 246 nm) to give 6,7-dimethoxy-4-(4-pyrrolidin-1-yl-1H-indazol-1-yl)cinnoline as a brown solid. LC/MS M+H 376.2.

### Example 16

### Synthesis of 4-(6,7-dimethoxycinnolin-4-yl)-6-(3-methoxyphenyl)morpholin-3-one

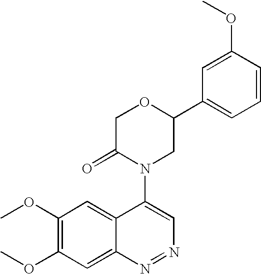

Into a 5 mL microwave tube was added 4-bromo-6,7-dimethoxycinnoline (63.6 mg, 0.236 mmol), 6-(3-methoxyphenyl)morpholin-3-one (41.7 mg, 0.201 mmol), copper(I) iodide (5.7 mg, 0.030 mmol), potassium carbonate (68.8 mg, 0.498 mmol), N,N′-dimethyl-1,2-ethanediamine (10 μL, 0.1 mmol) and tetrahydrofuran (0.3 mL, 0.004 mol). The reaction mixture was heated at 115° C. for 18.0 hours, filtered through celite rinsing with methylene chloride (20 mL) and concentrated (rotovap). The compound was purified on a C18 preparative HPLC column (30×100 mm) using acetonitrile in water (with 0.1% formic acid) in a gradient fashion going from 20% CH₃CN to 80% CH₃CN with a flow rate of 45 mL/min. Detection was performed at wavelength 325 nm and the product was collected from 4.8 to 5.0 minutes. The material was loaded onto an SCX column, rinsed with one column volume of MeOH and eluted with 2.0 M ammonia in methanol (10 mL). Removal of the solvent (rotovap) and drying under reduced pressure provided 9.9 mg of 4-(6,7-dimethoxycinnolin-4-yl)-6-(3-methoxyphenyl)morpholin-3-one as a light yellow solid.

### Example 17

### Synthesis of 7-methoxy-4-[(2S)-2-(4-methoxyphenyl)morpholin-4-yl]-6-(2,2,2-trifluoroethoxy)cinnoline

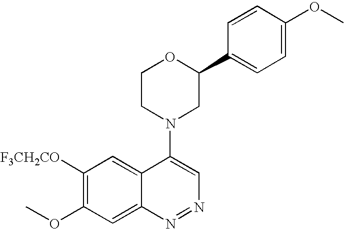

Step 1. A mixture of 6-(benzyloxy)-7-methoxy-4-[2-(4-methoxyphenyl)morpholin-4-yl]cinnoline (70.0 mg, 0.153 mmol), trifluoroacetic acid (5.00 mL, 64.9 mmol) and anisole (0.500 mL, 4.60 mmol) was sealed in a microwave tube and heated to 100° C. for 16 hours. The solvent was evaporated under vacuum and the residue was treated with 3 mL of 2M KOH in 85% MeOH and stirred at room temperature for 3 hours. The pH was adjusted to 6 by the addition of acetic acid. The resulting mixture was diluted with 30 mL of EtOAc and 3 mL of water, stirred vigorously for 5 minutes and the organic phase was separated and concentrated. The product was purified by column chromatography using a gradient elution going from 5 to 10% MeOH in DCM to give 6-hydroxy-7-methoxy-4-[2-(4-methoxyphenyl)morpholin-4-yl]cinnoline as a brown gum 0.040 mg (yield 71.2%).

Step 2. Sodium hydride (0.0072 g, 0.18 mmol) was added to a solution of 7-methoxy-4-[2-(4-methoxyphenyl)morpholin-4-yl]cinnolin-6-ol (0.055 g, 0.15 mmol) in DMA (3.0 mL). The reaction mixture was stirred at room temperature for one hour and 2-iodo-1,1,1-trifluoroethane (0.038 g, 0.18 mmol) was added. The reaction mixture stirred at ambient temperature for 8 hours and was then warmed to 70° C. for 72 hours. LC/MS showed ˜10% conversion and 10 mL of sodium bicarbonate and 30 mL of EtOAc were added and stirring continued for 5 minutes. The organic phase was separated, concentrated and purified by HPLC (prep1060, uv 267, rt 4.88 min) followed by column chromatography (8-12% MeOH/EtOAc) to give 7-methoxy-4-[(2S)-2-(4-methoxyphenyl)morpholin-4-yl]-6-(2,2,2-trifluoroethoxy)cinnoline as a yellow gum. LC/MS M+H 450.1.

### Example 18

### Synthesis of 1-{[1-(6,7-dimethoxycinnolin-4-yl)piperidin-3-yl]methyl}pyrrolidin-2-one

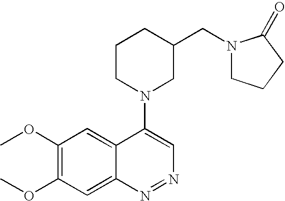

Into a flame-dried 5 mL microwave tube under argon was added 4-bromo-6,7-dimethoxycinnoline (99.2 mg, 0.369 mmol), 1-(piperidin-3-ylmethyl)pyrrolidin-2-one (50.7 mg, 0.278 mmol), tris(dibenzylideneacetone)dipalladium(0) (13.9 mg, 0.0152 mmol), 9,9-dimethyl-4,5-bis(diphenylphosphino)xanthene (17.4 mg, 0.0301 mmol), sodium tert-butoxide (41.1 mg, 0.428 mmol) and toluene (0.7 mL, 0.006 mol). The resulting yellow-brown suspension was warmed to 50° C. for 20.0 hours with stirring, cooled to room temperature for 2-3 hours and filtered through celite rinsing with 30 mL of 10% MeOH in DCM. The reaction mixture was concentrated and purified by preparative HPLC on a C18 column (30×100 mm) using 15% CH₃CN and 85% water with 0.1% formic acid for 4.0 min, followed by a gradient going from 15% CH₃CN to 80% CH₃CN in water with 0.1% formic acid with a flow rate of 45 mL/min. Detection was performed at a wavelength of 387 nm and the product was collected from 2.25 to 3.0 minutes. The material was loaded onto an SCX column (0.60 g), washed with one column volume of MeOH. (yellow band at top of column), eluted with 2.0 M ammonia in methanol (8 mL) and concentrated. The product was further purified on a Berger SFC Mini-Gram using a 10.0 mm×250 mm pyridine column using 15.0% MeOH with 0.1% DME in CO₂(1) as eluant with a flow rate of 9.9 mL/min and total run time of 5.0 minutes. 80 uL injections were run in sequence until all material was consumed. Collection was performed at wavelength=240 nm and the product had a retention time of 3.0 to 3.6 minutes to provide 26.3 mg of 1-{[1-(6,7-dimethoxycinnolin-4-yl)piperidin-3-yl]methyl}pyrrolidin-2-one as a yellow solid. LC/MS: M+H+371.2.

### Synthesis 19

### Synthesis of 6,7-dimethoxy-4-[2-(4-methoxyphenyl)-3-methylmorpholin-4-yl]cinnoline

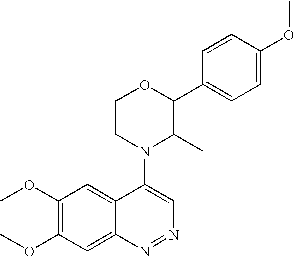

Into a 25 mL round bottom flask was added 4-bromo-6,7-dimethoxycinnoline (150 mg, 0.56 mmol), 2-(4-methoxyphenyl)-3-methylmorpholine (140 mg, 0.67 mmol), tris(dibenzylideneacetone)dipalladium(0) (26 mg, 0.028 mmol), 9,9-dimethyl-4,5-bis(diphenylphosphino)xanthene (32 mg, 0.056 mmol), sodium tert-butoxide (80 mg, 0.84 mol) and toluene (2 mL). The yellow-brown suspension was stirred for 24 hours at 55° C., and then flushed through an SCX column with methanol and eluted with 2.0 M ammonia in methanol. The material was purified by rotary chromatography using a gradient elution going from 100% chloroform to 10% methanol in chloroform to provide 6,7-dimethoxy-4-[2-(4-methoxyphenyl)-3-methylmorpholin-4-yl]cinnoline as a reddish foam.

### Example 20

### Synthesis of 1-(6,7-dimethoxycinnolin-4-yl)-N-(4-methoxybenzyl)piperidin-4-amine

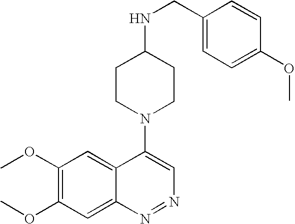

1-(6,7-Dimethoxycinnolin-4-yl)piperidin-4-amine (0.12 g, 0.42 mmol), 2 mL of methylene chloride and 4-methoxybenzaldehyde (0.085 g, 0.62 mmol) were combined and stirred at room temperature for 30 minutes followed by the addition of sodium cyanoborohydride (0.08 g, 1 mmol). The resulting mixture was stirred overnight and then purified on a 12 g silica gel column using a gradient elution going from 100% CH₂Cl₂ to 50% (8:1:1 CH₂Cl₂/MeOH/7M NH₃ in MeOH)/CH₂Cl₂ to provide 1-(6,7-dimethoxy-cinnolin-4-yl)-N-(4-methoxybenzyl)piperidin-4-amine.

### Biological Examples

### Example 21

### mPDE10A7 Enzyme Activity and Inhibition

Enzyme Activity: To analyze the enzyme activity, 5 μL of serial diluted mPDE10A7 containing lysate were incubated with equal volumes of diluted (100-fold) fluorescein labeled cAMP or cGMP for 30 minutes in MDC HE 96-well assay plates at room temperature. Both the enzyme and the substrates were diluted in the following assay buffer: Tris/HCl (pH 8.0) 50 mM, MgCl₂ 5 mM, 2-mercaptoethanol 4 mM, BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP Assay kit (Molecular Device) was used to assess enzyme properties of mPDE10A7. Data were analyzed with SoftMax Pro.

Enzyme Inhibition: To check the inhibition profile, 10 μL of serial diluted compounds were incubated with 30 μl of diluted PDE enzymes in a 96-well polystyrene assay plate for 30 minutes at room temperature. After incubation, 5 μL of the compound-enzyme mixture were aliquoted into a MDC HE black plate, mixed with 5 μl of 100-fold diluted fluorescein labeled substrates (cAMP or cGMP), and incubated for 30 minutes at room temperature. The reaction was stopped by adding 20 μL of diluted binding reagents and counted in an Analyst GT for fluorescence polarization. The data were analyzed with SoftMax Pro. The IC₅₀ values of representative compounds of this invention are shown in Tables 3 and 4 below.

TABLE 3

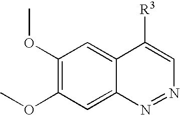

| Cpd | | mPDE10A7, |
|---|---|---|
| # | R³ | IC₅₀ (nM) |
| 1 | benzoxazol-2-yl | 218.36 |
| 3 | 3-cyclopropylaminocarbonyl-1H-indazol-1-yl | 140.56 |
| 8 | 4-methyl-3,4-dihydro-2H-benzo[b][1,4]oxazin-7-yl | 23.01 |
| 12 | 3-(RS)-phenylpyrrolidin-1-yl | 261.49 |
| 19 | 6-(2-methoxyethyloxy)-1H-indazol-1-yl | 139.85 |
| 21 | 6-(morpholin-4-yl)-1H-indazol-1-yl | 554.06 |
| 32 | 3-(RS)-(pyrrolidin-1-ylcarbonyl)piperidin-1-yl | 402.48 |
| 37 | 3-(RS)-(phenoxy)piperidin-1-yl | 113.46 |
| 38 | 2-(RS)-(2,3-dihydrobenzofuran-5-yl)morpholin-4-yl | 6.49 |
| 39 | 3-(RS)-(piperidin-1-ylcarbonyl)piperidin-1-yl | 378 |
| 40 | 2-(RS)-(4-chlorophenyl)-5-oxo-morpholin-4-yl | 426.98 |
| 45 | 1-acetyl-3-(RS)-(phenyl)piperazin-1-yl | 408.84 |
| 48 | 4-(1-ethylpiperazin-4-yl)1-1H-indazol-1-yl | 28.78 |
| 50 | 4-(1-cyclopropylmethylpiperazin-4-yl)1-1H-indazol- | 63.11 |
| | 1-yl | |
| 51 | 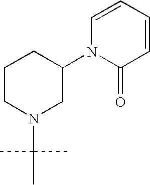 | 151.72 |
| 52 | 2-(RS)-(3-methoxyphenyl)-3-oxomorpholin-4-yl | 310.04 |
| 56 | 3-(RS)-(3-methyl-[1.2.4]oxadiazol-5-yl)piperidin-1- | 189.75 |
| | yl | |
| 62 | 3-(RS)-(2-oxopyrrolidin-1-ylmethyl)piperidin-1-yl | 336.62 |
| 63 | 2-(RS)-(4-trifluoromethoxyphenyl)morpholin-4-yl | 29.24 |
| 64 | 3-hydroxy-3-(4-methoxyphenyl)piperidin-1-yl | 19.17 |
| 65 | 6-oxo-2-(RS)-(4-methoxyphenyl)-1- | 46.11 |
| | methylpiperazin-4-yl | |
| 67 | 3-(S)-(2-fluorophenyl)piperidin-1-yl | 7.01 |
| 70 | 1-(methyoxycarbonylmethyl)-3-(RS)-(3- | 7.21 |
| | methoxyphenyl)-piperazin-1-yl | |
| 72 | 4-ethylmethylamino-1H-indazol-1-yl | 43.99 |
| 73 | 4-pyrrolidin-1-yl-1H-indazol-1-yl | 96.15 |
| 74 | 2-(RS)-(4-hydroxyphenyl)morpholin-4-yl | 3.84 |
| 76 | 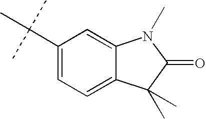 | 18.73 |
| 78 | 4-(RS)-(4-methoxybenzylamino)piperidin-1-yl | 1394.22 |
| 85 | 4-(RS)-(2-oxopyrrolidin-1-yl)piperidin-1-yl | 285.78 |
| 86 | 2-(RS)-(6-methoxynaphth-2-yl)piperazin-4-yl | 1.33 |
| 88 | 2-(RS)-(2-fluoro-4-methylphenyl)morpholin-4-yl | 57.78 |
| 93 | 2-(RS)-(thiophen-2-yl)piperazin-4-yl | 7.84 |
| 94 | 2-(RS)-(4-dimethylaminophenyl)piperazin-4-yl | 1.6 |
| 96 | 2-(RS)-(2-methylphenyl)morpholin-4-yl | 14.05 |
| 98 | 3-(RS)-(4-methoxybenzyl)pyrrolidin-1-yl | 26.57 |
| 100 | 2-(RS)-ethyl-6-(RS)-(4-methoxyphenyl)morpholin- | 3.57 |
| | 4-yl | |
| 101 | 1-(4-methoxyphenyl)-1,2,5,6-tetrahydropyridin-3-yl | 6.51 |
| 104 | 3-(RS)-(cyclohexyl)piperidin-1-yl | 263.38 |
| 107 | 3-(RS)-(—N(CH₃)COphenyl)piperidin-1-yl | 58.42 |
| 110 | 2-(S)-(2-fluoro-4-methylphenyl)morpholin-4-yl | 2.86 |
| 111 | 2-(R)-(6-methoxynaphth-2-yl)piperazin-4-yl | 10.35 |
| 113 | 4-bromo-1H-indazol-1-yl | 152.01 |
| 106 | 4-(1-acetylpiperazin-4-yl)-1H-indazol-1-yl | 15.97 |
| 118 | 2-(RS)-(3,5-dibenzyloxyphenyl)piperazin-4-yl | 159.82 |
| 121 | 3-(RS)-(3,5-dimethoxyphenylcarbonylamino)- | 16.34 |
| | piperidin-1-yl | |
| 102 | 3-(RS)-(2-fluorophenylcarbonylamino)-piperidin- | 35.09 |
| | 1-yl | |
| 103 | 2-(RS)-(naphth-1-yl)piperazin-4-yl | 12.04 |

TABLE 4

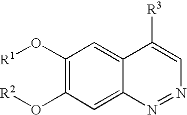

| CPD | | | | mPDE10A7 |
|---|---|---|---|---|
| # | R¹ | R² | R³ | IC₅₀ (nM) |
| 125 | —CH₂CF₃ | methyl | 2-(RS)-(4-methoxy- | 1191.37 |
| | | | phenyl)morpholin-4-yl | |
| 127 | methyl | H | 2-(RS)-(4-methoxy- | 144.91 |
| | | | phenyl)morpholin-4-yl | |

### Example 22

### Apomorphine Induced Deficits in Prepulse Inhibition of the Startle Response in Rats, an in vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g. the reflex activity of the rats is measured. The reflex of the rats to the burst of noise may be attenuated by preceding the startle stimulus with a stimulus of lower intensity, at 3 to 12 db above background (65 db), which will attenuate the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine will reduce the inhibition of the startle reflex produced by the prepulse. Antipsychotic drugs such as haloperidol will prevent apomorphine from reducing the prepulse inhibition of the startle reflex. This assay may be used to test the antipsychotic efficacy of PDE10 inhibitors. Representative compounds provided herein were tested and determined to reduce the apomorphine-induced deficit in the prepulse inhibition of startle.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed:

1. A compound of Formula (I): 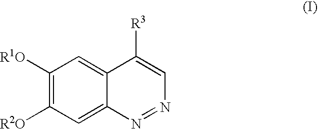
wherein:
R¹ and R² are independently selected from hydrogen, alkyl, or haloalkyl; and
R³ is:
(i) a ring of formula (a) 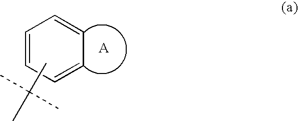
where A is a monocyclic five-, six-, or seven membered heterocyclyl ring and the ring of formula (a) is substituted with:
R⁴ where R⁴ is hydrogen, cycloalkyl, cycloalkylalkyl, aryl, heteroaryl, heterocyclyl, aralkyl, heteroaralkyl, heterocyclylalkyl, or —XR⁷ (where X is —O—, —CO—, —C(O)O—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
R⁵ and R⁶ where R⁵ and R⁶ are independently hydrogen, alkyl, cycloalkyl, cycloalkylalkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl;
and wherein the aromatic or alicyclic ring in R⁴, R⁵, R⁶, and R⁷ is optionally substituted with one to three substitutents independently selected from R^{a}, R^{b}, and R^{c} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are independently hydrogen or fluoro;
(ii) a ring of formula (b) or (c): 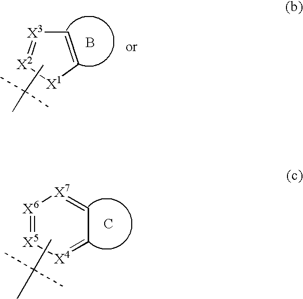
where:
X¹, X², and X³ are independently carbon, nitrogen, oxygen or sulfur provided that at least two of X¹, X², and X³ are other than carbon;
X⁴, X⁵, X⁶ and X⁷ are independently carbon or nitrogen provided that at least two of X⁴, X⁵, X⁶ and X⁷ are other than carbon; and
B and C are phenyl, a five- or six-membered heteroaryl ring (wherein the five-membered heteroaryl ring contains one or two heteroatoms independently selected from nitrogen, oxygen, and sulfur and the six-membered heteroaryl ring contains one or two nitrogen atoms, the rest of the ring atoms being carbon), or a monocyclic five-, six-, or seven-membered heterocyclyl ring; and
wherein rings of formulae (b) and (c) are substituted with:
R¹³ where R¹³ is hydrogen, cycloalkyl, cycloalkylalkyl, aryl, heteroaryl, heterocyclyl, aralkyl, heteroaralkyl, heterocyclylalkyl, or —XR¹⁶ (where X is —O—, —CO—, —C(O)O—, —NR¹⁷CO—, —CONR¹⁸—, —NR¹⁹—, —S—, —SO—, —SO₂—, —NR²⁰SO₂—, or —SO₂NR²¹— where R¹⁷, R¹⁸, R¹⁹, R²⁰ and R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl); and
R¹⁴ and R¹⁵ where R¹⁴ and R¹⁵ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and
wherein the aromatic or alicyclic ring in R¹³, R¹⁴, R¹⁵, and R¹⁶ is optionally substituted with one to three substitutents independently selected from R^{f}, R^{g}, and R^{h} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkylcarbonyl, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁱ and R^{j} where Rⁱ and R^{j} are independently hydrogen or fluoro;
(iii) a monocyclic six- or seven-membered heterocyclyl ring substituted with:
R²² where R²² is cycloalkyl, aryl, heteroaryl, heterocyclyl, aralkyl, heteroaralkyl, heterocyclylalkyl, or —XR²⁵ (where X is —O—, —CO—, —C(O)O—, —NR²⁶CO—, —CONR²⁷—, —NR²⁸—, —S—, —SO—, —SO₂—, —NR²⁹SO₂—, or —SO₂NR³⁰— where R²⁶, R²⁷, R²⁸, R²⁹ and R³⁰ are independently hydrogen, alkyl, hydroxyalkyl, alkoxyalkyl, aryl, aralkyl, heteroaryl, heteroaralkyl, acyl, or heterocyclylalkyl and R²⁵ is cycloalkyl, cycloalkylalkyl, aryl, heteroaryl, heterocyclyl, aralkyl, heteroaralkyl, or heterocyclylalkyl); and
R²³ and R²⁴ where R²³ and R²⁴ are independently hydrogen, alkyl, cycloalkyl, cycloalkylalkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkoxycarbonylalkyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and
wherein the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, amino, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are independently hydrogen or fluoro; or
(iv) pyrrolidinyl, 2-oxopyrrolidinyl, or 2,4-dioxoimidazolidinyl substituted with:
R³¹ where R³¹ is aryl, heteroaryl, heterocyclyl, aralkyl, heteroaralkyl, heterocyclylalkyl, or —XR³⁴ (where X is —O—, —CO—, —C(O)O—, —NR³⁵CO—, —CONR³⁶—, —NR³⁷—, —S—, —SO—, —SO₂—, —NR³⁸SO₂—, or —SO₂NR³⁹— where R³⁵, R³⁶, R³⁷, R³⁸ and R³⁹ are independently hydrogen, alkyl, hydroxyalkyl, alkoxyalkyl, aryl, aralkyl, heteroaryl, heteroaralkyl, acyl, or heterocyclylalkyl and R³⁴ is cycloalkyl, cycloalkylalkyl, aryl, heteroaryl, heterocyclyl, aralkyl, heteroaralkyl, or heterocyclylalkyl); and
R³² and R³³ where R³² and R³³ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, aryl, heteroaryl or heterocyclyl; and
wherein the aromatic or alicyclic ring in R³¹, R³², R³³, and R³⁴ is optionally substituted with one to three substitutents independently selected from R^{p}, R^{q}, and R^{r} which are independently alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, carboxy, alkoxycarbonyl, sulfonyl, aminocarbonyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{s} and R^{t} where R^{s} and R^{t} are independently hydrogen or fluoro;
an individual stereoisomer, mixtures of stereoisomers, or a pharmaceutically acceptable salt thereof, provided that:
(i) the compound of Formula (I) is not 4-(4-(3-chlorophenyl)piperazin-1-yl)-6,7-dimethoxycinnoline; 4-(4-(benzo[d]isothiazol-3-yl)piperazin-1-yl)-6,7-dimethoxycinnoline;
(ii) when R³ is pyrrolidin-1-yl, R³¹ is not —XR³⁴ where X is —O— and R³⁴ is substituted or unsubstituted aryl or heteroaryl;
(iii) when R³ is piperidin-1-yl, one of R²³ and R²⁴ is hydrogen, and R²² is substituted or unsubstituted aryl or heteroaryl, then the other of R²³ and R²⁴ is not hydrogen, alkyl, carboxy, alkoxycarbonyl, cyano, hydroxyl, alkoxy, —COR, —CONRR′ or —NRR′ (where R and R′ are independently hydrogen, alkyl, or unsubstituted aryl), or —NHCOR (where R is alkyl or unsubstituted aryl); and
(iv) when R³ is piperidin-1-yl, R²³ and R²⁴ are both hydrogen or one of R²³ and R²⁴ is hydrogen and the other of R²³ and R²⁴ is substituted or unsubstituted aryl or heteroaryl, then R²² is not —COR²⁵ (where R²⁵ is unsubstituted aryl), —COOR²⁵ (where R²⁵ is unsubstituted aryl), —CONR²⁵R²⁷, —NR²⁵R²⁸ or —NHCOR²⁵ (where R²⁷ and R²⁸ are hydrogen, alkyl, or unsubstituted aryl, and each R²⁵ is unsubstituted aryl).

2. The compound of claim 1 wherein R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl;
and wherein the aromatic or alicyclic ring in R⁴, R⁵, R⁶, and R⁷ is optionally substituted with one to three substitutents independently selected from R^{a}, R^{b}, and R^{c} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are independently hydrogen or fluoro;
R¹³ where R¹³ is hydrogen, cycloalkyl, cycloalkylalkyl, aryl, heteroaryl, heterocyclyl, aralkyl, heteroaralkyl, heterocyclylalkyl, or —XR¹⁶ (where X is —O—, —CO—, —C(O)O—, —NR¹⁷CO—, —CONR¹⁸—, —NR¹⁹—, —S—, —SO—, —SO₂—, —NR²⁰SO₂—, or —SO₂NR²¹— where R¹⁷, R¹⁸, R¹⁹, R²⁰ and R²¹ are independently hydrogen, alkyl, hydroxyalkyl, alkoxyalkyl, aryl, aralkyl, heteroaryl, heteroaralkyl, acyl, or heterocyclylalkyl and R¹⁶ is cycloalkyl, cycloalkylalkyl, aryl, heteroaryl, heterocyclyl, aralkyl, heteroaralkyl, or heterocyclylalkyl); and
R¹⁴ and R¹⁵ where R¹⁴ and R¹⁵ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and
wherein the aromatic or alicyclic ring in R¹³, R¹⁴, R¹⁵, and R¹⁶ is optionally substituted with one to three substitutents independently selected from R^{f}, R^{g}, and R^{h} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁱ and R^{j} where Rⁱ and R^{j} are independently hydrogen or fluoro; and
R²³ and R²⁴ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl; and
wherein the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are independently hydrogen or fluoro.

3. The compound of claim 2 wherein R¹ and R² are alkyl and R³ is a ring of formula (a).

4. The compound of claim 2 wherein R¹ and R² are alkyl and R³ is a monocyclic six- or seven-membered heterocyclyl ring substituted with R²², R²³ and R²⁴ wherein the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are independently hydrogen or fluoro.

5. The compound of claim 4 wherein R³ is a ring of formula: 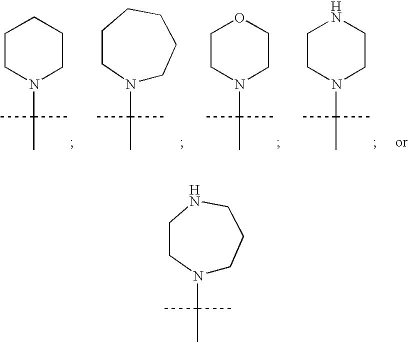
substituted, including the —NH— groups in the rings, with R²², R²³ and R²⁴ wherein the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl.

6. The compound of claim 4 wherein R³ is a ring of formula: 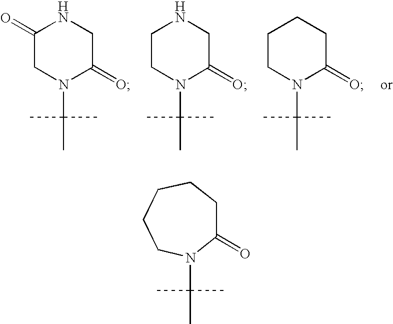
substituted, including the —NH— groups in the rings, with R²², R²³ and R²⁴ wherein the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl.

7. The compound of claim 4 wherein R³ is a ring of formula: 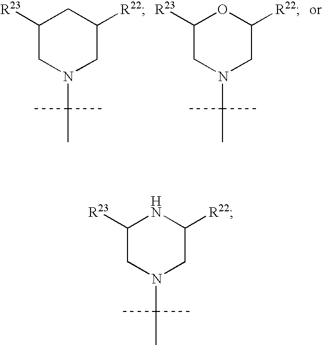
where:
R²² is phenyl or heteroaryl, each substituted at the para position with R^{k} and optionally substituted with R^{l} and R^{m}.

8. The compound of claim 7 wherein R²³ is hydrogen.

9. The compound of claim 4 wherein R³ is a ring of formula: 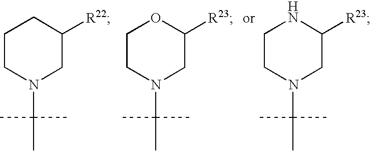
where:
R²² is phenyl or heteroaryl, each substituted with R^{k} and R^{l}.

10. The compound of claim 9 where R²² is phenyl substituted with R^{k} and R^{l}.

11. The compound of claim 9 where R²² is heteroaryl substituted with R^{k} and R^{l}.

12. The compound of claim 2 wherein R¹ and R² are alkyl and R³ is a ring of formula (b) substituted with R¹³, R¹⁴, and R¹⁵.

13. The compound of claim 2 wherein R¹ is haloalkyl, R² are alkyl, and R³ is a monocyclic six- or seven-membered heterocyclyl ring substituted with R²², R²³ and R²⁴ wherein the aromatic or alicyclic ring in R²², R²³, R²⁴, and R²⁵ is optionally substituted with one to three substitutents independently selected from R^{k}, R^{l}, and R^{m} which are independently alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from Rⁿ and R^{o} where Rⁿ and R^{o} are independently hydrogen or fluoro.

14. The compound of claim 12 wherein R³ is a a ring of formula:

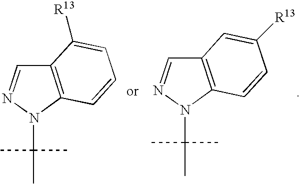

15. The compound of claim 1 wherein R¹ and R² are alkyl and R³ is monocyclic six- or seven-membered heterocyclyl ring substituted with R²² where R²² is aryl, heteroaryl, heterocyclyl, aralkyl, heterocyclylalkyl, or —XR²⁵ (where X is —O—, —CO—, —NH⁶CO—, or —NH— where R²⁵ is aryl, heterocyclyl, or aralkyl); and
R²³ where R²³ is selected from hydrogen, alkyl, hydroxyl, or acyl; and
wherein the aromatic or alicyclic ring in R²², R²³, and R²⁵ is optionally substituted with one to three substitutents independently selected from R^{k}, R^{l}, and R^{m} which are independently alkyl, alkoxy, halo, haloalkoxy, hydroxyl, cyano, and disubstituted amino; or
R³ is pyrrolidin-1-yl substituted with R³¹ where R³¹ is aryl, aralkyl, or —XR³⁴ (where X is —NHCO—, or —NH— where R³⁴ is aryl or aralkyl wherein the aromatic ring in R³¹ is optionally substituted with one to three substitutents independently selected from R^{p}, R^{q}, and R^{r} which are alkoxy.

16. A compound selected from Tables 1 and 2 below:
TABLE 1

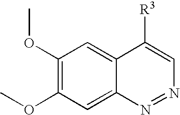

| Cpd # | R³ |
|---|---|
| 1 | benzoxazol-2-yl |
| 2 | indazol-1-yl |
| 3 | 3-cyclopropylaminocarbonyl-1H-indazol-1-yl |
| 4 | 2-(RS)-phenylmorpholin-4-yl |
| 5 | 2,3-dihydrobenzo[b][1,4]dioxin-6-yl |
| 6 | 5-benzyloxy-1H-indazol-1-yl |
| 7 | 6-benzyloxy-1H-indazol-1-yl |
| 8 | 4-methyl-3,4-dihydro-2H-benzo[b][1,4]oxazin-7-yl |
| 9 | 2-(R)-phenylmorpholin-4-yl |
| 10 | 2-(S)-phenylmorpholin-4-yl |
| 11 | 2-(RS)-(4-methoxyphenyl)morpholin-4-yl |
| 12 | 3-(RS)-phenylpyrrolidin-1-yl |
| 13 | 2-(RS)-(4-fluorophenyl)morpholin-4-yl |
| 14 | 2-(RS)-(2-chlorophenyl)morpholin-4-yl |
| 15 | 2-(RS)-(pyridin-3-yl)morpholin-4-yl |
| 16 | 4-(RS)-(phenoxy)piperidin-1-yl |
| 17 | 4-(2-methoxyethyloxy)-1H-indazol-1-yl |
| 18 | 5-(2-methoxyethyloxy)-1H-indazol-1-yl |
| 19 | 6-(2-methoxyethyloxy)-1H-indazol-1-yl |
| 20 | 5-(morpholin-4-yl)-1H-indazol-1-yl |
| 21 | 6-(morpholin-4-yl)-1H-indazol-1-yl |
| 22 | 5-(pyridin-3-yl)-1H-indazol-1-yl |
| 23 | 5-(pyridin-4-yl)-1H-indazol-1-yl |
| 24 | 4-(pyridin-4-yl)-1H-indazol-1-yl |
| 25 | 4-(morpholin-4-yl)-1H-indazol-1-yl |
| 26 | 4-(4-methylpiperazin-1-yl)-1H-indazol-1-yl |
| 27 | 2-(RS)-(pyrrolidin-1-ylmethyl)morpholin-4-yl |
| 28 | 3-(RS)-(2-oxopiperidin-1-yl)piperidin-1-yl |
| 29 | 2-(RS)-(benzyl)pyrrolidin-1-yl |
| 30 | 4-methyl-3-(RS)-(phenyl)piperazin-1-yl |
| 31 | 1,3-benzodioxol-5-yl |
| 32 | 3-(RS)-(pyrrolidin-1-ylcarbonyl)piperidin-1-yl |
| 33 | 3-(RS)-benzylpiperidin-1-yl |
| 34 | 3-(R)-(2-oxopiperidin-1-yl)piperidin-1-yl |
| 35 | 3-(S)-(2-oxopiperidin-1-yl)piperidin-1-yl |
| 36 | 3-(RS)-indol-1-ylpiperidin-1-yl |
| 37 | 3-(RS)-(phenoxy)piperidin-1-yl |
| 38 | 2-(RS)-(2,3-dihydrobenzofuran-5-yl)morpholin-4-yl |
| 39 | 3-(RS)-(piperidin-1-ylcarbonyl)piperidin-1-yl |
| 40 | 2-(RS)-(4-chlorophenyl)-5-oxo-morpholin-4-yl |
| 41 | 2-(S)-(4-methoxyphenyl)morpholin-4-yl |
| 42 | 2-(R)-(4-methoxyphenyl)morpholin-4-yl |
| 43 | 3-(RS)-(2-fluorophenyl)piperidin-1-yl |
| 44 | 3-(RS)-(phenyl)piperazin-1-yl |
| 45 | 1-acetyl-3-(RS)-(phenyl)piperazin-1-yl |
| 46 | 4-(piperazin-1-yl)1-1H-indazol-1-yl |
| 47 | 2-(RS)-(4-fluorophenyl)-2-methylmorpholin-4-yl |
| 48 | 4-(1-ethylpiperazin-4-yl)1-1H-indazol-1-yl |
| 49 | 4-(1-methyl-2-oxo-piperazin-4-yl)1-1H-indazol-1-yl |
| 50 | 4-(1-cyclopropylmethylpiperazin-4-yd)1-1H-indazol-1-yl |
| 51 | 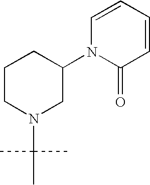 |
| 52 | 2-(RS)-(3-methoxyphenyl)-3-oxomorpholin-4-yl |
| 53 | 2-(RS)-(3-methoxyphenyl)morpholin-4-yl |
| 54 | 2-(RS)-(2-methoxyphenyl)piperidin-1-yl |
| 55 | 3-(RS)-(3-methoxyphenyl)piperazin-1-yl |
| 56 | 3-(RS)-(3-methyl-[1.2.4]oxadiazol-5-yl)piperidin-1-yl |
| 57 | 2-(RS)-(4-chlorophenyl)morpholin-4-yl |
| 58 | 2-(RS)-(4-methylphenyl)morpholin-4-yl |
| 59 | 2-(RS)-(3,5-dichlorophenyl)morpholin-4-yl |
| 60 | 2-(RS)-(3-iodo-4-methoxyphenyl)morpholin-4-yl |
| 61 | 6-methyl-2-(RS)-(4-methoxyphenyl)morpholin-4-yl |
| 62 | 3-(RS)-(2-oxopyrrolidin-1-ylmethyl)piperidin-1-yl |
| 63 | 2-(RS)-(4-trifluoromethoxyphenyl)morpholin-4-yl |
| 64 | 3-hydroxy-3-(4-methoxyphenyl)piperidin-1-yl |
| 65 | 6-oxo-2-(RS)-(4-methoxyphenyl)-1-methylpiperazin-4-yl |
| 66 | 5-(4-methoxyphenyl)-1,2,3,4-tetrahydropyridin-1-yl |
| 67 | 3-(S)-(2-fluorophenyl)piperidin-1-yl |
| 68 | 3-(R)-(2-fluorophenyl)piperidin-1-yl |
| 69 | 2-(RS)-(4-methoxyphenyl)-1-methylpiperazin-4-yl |
| 70 | 1-(methyoxycarbonylmethyl)-3-(RS)-(3- |
| | methoxyphenyl)piperazin-1-yl |
| 71 | 4-dimethylamino-1H-indazol-1-yl |
| 72 | 4-ethylmethylamino-1H-indazol-1-yl |
| 73 | 4-pyrrolidin-1-yl-1H-indazol-1-yl |
| 74 | 2-(RS)-(4-hydroxyphenyl)morpholin-4-yl |
| 75 | 3-(RS)-(4-methoxybenzylamino)pyrrolidin-1-yl |
| 76 | 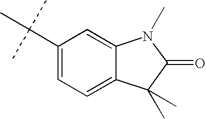 |
| 77 | 2-(RS)-(4-methoxyphenyl)-3-methylmorpholin-4-yl |
| 78 | 4-(RS)-(4-methoxybenzylamino)piperidin-1-yl |
| 79 | 3-(RS)-(3,5-dimethoxyphenyl)piperazin-4-yl |
| 80 | 4-(1-ethylpiperazin-4-yl)-1H-indazol-1-yl |
| 81 | 3-(RS)-1-(4-methoxyphenyl)piperidin-3-yl |
| 82 | 3-(RS)-(2-oxopyrrolidin-1-yl)piperidin-1-yl |
| 83 | 4-(RS)-(2-oxopiperidin-1-yl)piperidin-1-yl |
| 84 | 4-(RS)-(2-oxoazetidin-1-yl)piperidin-1-yl |
| 85 | 4-(RS)-(2-oxopyrrolidin-1-yl)piperidin-1-yl |
| 86 | 2-(RS)-(6-methoxynaphth-2-yl)piperazin-4-yl |
| 87 | 2-(RS)-(4-methoxyphenyl)piperazin-4-yl |
| 88 | 2-(RS)-(2-fluoro-4-methylphenyl)morpholin-4-yl |
| 89 | 2-(S)-(2-chlorophenyl)morpholin-4-yl |
| 90 | 2-(R)-(2-chlorophenyl)morpholin-4-yl |
| 91 | 2-(RS)-(2-cyanophenyl)morpholin-4-yl |
| 92 | 2-(RS)-(2,6-difluorophenyl)morpholin-4-yl |
| 93 | 2-(RS)-(thiophen-2-yl)piperazin-4-yl |
| 94 | 2-(RS)-(4-dimethylaminophenyl)piperazin-4-yl |
| 95 | 3-(RS)-(4-methoxyphenylcarbonylamino)pyrrolidin-1-yl |
| 96 | 2-(RS)-(2-methylphenyl)morpholin-4-yl |
| 97 | 3-(RS)-(naphth-2-yl)piperazin-1-yl |
| 98 | 3-(RS)-(4-methoxybenzyl)pyrrolidin-1-yl |
| 99 | 3-(RS)-(phenylcarbonylamino)piperidin-1-yl |
| 100 | 2-(RS)-ethyl-6-(RS)-(4-methoxyphenyl)morpholin-4-yl |
| 101 | 1-(4-methoxyphenyl)-1,2,5,6-tetrahydropyridin-3-yl |
| 102 | 3-(RS)-(2-fluorophenylcarbonylamino)piperidin-1-yl |
| 103 | 2-(RS)-(naphth-1-yl)piperazin-4-yl |
| 104 | 3-(RS)-(cyclohexyl)piperidin-1-yl |
| 105 | 4-(RS)-(phenyl)azepan-1-yl |
| 106 | 4-(4-acetylpiperazin-1-yl)-1H-indazol-1-yl |
| 107 | 3-(RS)-(—N(CH₃)COphenyl)piperidin-1-yl |
| 108 | 3-(RS)-(4-chlorophenylcarbonylamino)piperidin-1-yl |
| 109 | 2-(R)-(2-fluoro-4-methylphenyl)morpholin-4-yl |
| 110 | 2-(S)-(2-fluoro-4-methylphenyl)morpholin-4-yl |
| 111 | 2-(R)-(6-methoxynaphth-2-yl)piperazin-4-yl |
| 112 | 2-(S)-(6-methoxynaphth-2-yl)piperazin-4-yl |
| 113 | 4-bromo-1H-indazol-1-yl |
| 114 | 2-(RS)-(2-hydroxypyridin-5-yl)morpholin-4-yl |
| 115 | 2-(RS)-(4-methoxyphenyl)-5(RS)-methylmorpholin-4-yl |
| 116 | 2-(RS)-(3,4-dimethoxyphenyl)morpholin-4-yl |
| 117 | 2-(RS)-(2-fluoro-4-methoxyphenyl)morpholin-4-yl |
| 118 | 2-(RS)-(3,5-dibenzyloxyphenyl)piperazin-4-yl |
| 119 | 2-(S)-(naphth-2-yl)piperazin-4-yl |
| 120 | 2-(R)-(naphth-2-yl)piperazin-4-yl |
| 121 | 3-(RS)-(3,5-dimethoxyphenylcarbonylamino)piperidin-1-yl |

TABLE 2

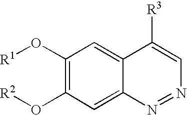

| CPD # | R¹ | R² | R³ |
|---|---|---|---|
| 122 | ethyl | methyl | 2-(RS)-(4-methoxyphenyl)morpholin-4-yl |
| 123 | ethyl | methyl | 2-(R)-(4-methoxyphenyl)morpholin-4-yl |
| 124 | ethyl | methyl | 3-(RS)-(3-oxopiperidin-1-yl)piperidin-1-yl |
| 125 | —CH₂CF₃ | methyl | 2-(RS)-(4-methoxyphenyl)morpholin-4-yl |
| 126 | n-propyl | methyl | 2-(RS)-(4-methoxyphenyl)morpholin-4-yl |
| 127 | methyl | H | 2-(RS)-(4-methoxyphenyl)morpholin-4-yl |
| 128 | H | methyl | 2-(RS)-(4-methoxyphenyl)morpholin-4-yl |

or a pharmaceutically acceptable salt thereof.

17. A pharmaceutical composition comprising a compound of claim 1 and a pharmaceutically acceptable expicient.

18. A pharmaceutical composition comprising a compound of claim 16 and a pharmaceutically acceptable expicient.

19. A method of treating a disorder treatable by inhibition of PDE10 in a patient which method comprises administering to the patient a pharmaceutical composition comprising a compound of claim 1 and a pharmaceutically acceptable expicient.

20. A method of treating a disorder treatable by inhibition of PDE10 in a patient which method comprises administering to the patient a pharmaceutical composition comprising a compound of claim 16 and a pharmaceutically acceptable expicient.

21. The method of claim 19 wherein the disease is schizophrenia, bipolar disorder, or obsessive-compulsive disorder.
